(19) United States
(12) Patent Application Publication
(10) Pub. No.: US 2011/0245759 A1
(43) Pub. Date: Oct. 6, 2011

(54) Methods of Treating An Infection with Nitric Oxide

(75) Inventors: Frank J. McCaney, Voorhees, NJ (US); Alex Stenzler, Long Beach, CA (US)

(73) Assignees: NITRIC BIOTHERAPEUTICS, INC.; CARDINAL HEALTH 207, INC.

(21) Appl. No.: 13/055,427

(22) PCT Filed: Jul. 30, 2009
(86) PCT No.: PCT/US09/52268

§ 371 Date: Jun. 20, 2011

Related U.S. Application Data
(60) Provisional application No. 61/084,866, filed on Jul. 30, 2008.

(51) Int. Cl. A61M 37/00; A61K 33/00; A61P 17/02; A61P 31/12; A61P 31/10; A61P 31/04; A61P 33/02; A61P 33/00

(52) U.S. Cl. 604/24; 424/718

(57) ABSTRACT

The present invention provides methods of treating bacterial, viral, protozoan, parasitic, arthropod and fungal infections by applying gaseous nitric oxide to a patient having an infection. The present invention further provides improved methods of treating bacterial, viral, protozoan, parasitic and fungal infections by applying gaseous nitric oxide, in combination with oxygen, to a patient having an infection.

### BACKGROUND OF THE INVENTION

The treatment of surface or subsurface lesions infected with a pathogen, including bacteria, viruses, protozoa, and fungi, has typically involved the topical or systemic administration of one or more anti-infective agents to a patient. Antibiotics are one such class of anti-infective agents that are commonly used to treat an infected abscess, lesion, wound, or the like. Unfortunately, an increasing number of infectious agents, including bacteria, have become resistant to conventional antibiotic therapy. Indeed, the increased use of antibiotics by the medical community has led to a commensurate increase in resistant strains of bacteria that do not respond to traditional or even newly developed anti-bacterial agents. Even when new anti-infective agents are developed, these agents are extremely expensive, tend to have more side-effects than previously introduced antibiotics and are available only to a limited is patient population.

Staphylococcus aureus, for example, has developed resistance to many commonly used antibiotics including, chloramphenicol, rifampin, ciproflaxacin, clindamycin, erythromycin, beta-lactams, tetracycline, methicillin, vancomycin and trimethoprim. Methicillin-resistant Staphylococcus aureus (MRSA), also known as multiple-resistant Staphylococcus aureus and oxacillin-resistant Staphylococcus aureus (ORSA), has developed the ability to resist and survive treatment with beta-lactam antibiotics, including penicillin, methicillin, and cephalosporins. Vancomycin-resistant Staphylococcus aureus (VRSA) has developed the ability to resist and survive treatment vancomycin, a glycoprotein antibiotic. Vancomycin-resistant Staphylococcus aureus may also be methicillin resistant. Methicillin-resistant Staphylococcus aureus may also be vancomycin resistant. A total of 8987 cases of invasive MRSA infections were reported between July 2004 and December 2005, with the overall incidence rate of MRSA infections in 2005 estimated to be 31.8 in 100,000 persons (Klevens, 2007, JAMA 298:1763-1771). MRSA has been reported to be the leading cause of soft tissue infections, including skin infections, that led patients to seek medical attention in emergency rooms (Moran, 2006, N. Engl. J. Med. 355:666-674).

Pseudomonas aeruginosa, for example, displays resistance to many commonly used antibiotics. Pseudomonas aeruginosa is a relatively common nosocomial infection. According to the Centers for Disease Control and Prevention, the overall prevalence of Pseudomonas aeruginosa infections in US hospitals is approximately 4 per 1000 hospital discharges. Pseudomonas aeruginosa has a propensity to develop multiple-drug resistance (Paramythiotou, 2004, Clin. Infect. Dis. 38:670-7). Antibiotic-resistant Pseudomonas aeruginosa, sometimes known as multi-drug resistant Pseudomonas aeruginosa (MDRPA), can display decreased susceptibility to, for example, one or more of piperacillin, ceftazidime, imipenem, penicillin, aminoglycosides, beta-lactams, cephalosporin, quinolone, tetracycline, sulfonamides, chloramphenicol and ciprofloxacin (Livermore, 2001, J. Antimicrob. Chemother. 47:247-250; Pankey, 2005, Antimicrob. Agents Chemother. 49:2959-2964).

Streptococcus pneumoniae, for example, is known to display resistance to aminoglycosides, penicillin, chloramphenicol, erythromycin, trimethoprim and sulfamethoxazole. Enterobacteriae, including Escherichia coli, Salmonella, Klebsiella, are is known to display resistance to aminoglycosides, beta-lactams, chloramphenicol, and trimethoprim. Enterococci, for example, are known to display resistance to aminoglycosides, beta-lactams, erythromycin, and vancomycin.

Another problem with conventional anti-infective agents is that some patients are allergic to the very compounds necessary to their treat their infection. For these patients, only a few drugs might be available to treat the infection. If the patient is infected with a strain of bacteria that does not respond well to substitute therapies, the patient's life can be in danger.

A separate problem related to conventional treatment of surface or subsurface infections is that the infectious agent interferes with the circulation of blood within the infected region. It is sometimes the case that the infectious agent causes constriction of the capillaries or other small blood vessels in the infected region which reduces bloodflow. When bloodflow is reduced, a lower level of anti-infective agent than expected can be delivered to the infected region. In addition, the infection can take a much longer time to heal when bloodflow is restricted to the infected area. This increases the total amount of drug that must be administered to the patient, thereby increasing the cost and potential for unwanted side effects of using such drugs. Topical agents may sometimes be applied over the infected region. However, topical anti-infective agents sometimes do not penetrate deep within the skin where a significant portion of the bacteria often reside. Topical treatments of anti-infective agents are often less effective at eliminating infection than systemic administration (i.e., oral administration) of an anti-infective pharmaceutical.

In the 1980's, it was discovered by researchers that the endothelium tissue of the human body produced nitric oxide (NO), and that nitric oxide is an endogenous vasodilator, namely, an agent that widens the internal diameter of blood vessels. Prior to the 1980's, nitric oxide was most commonly known as an environmental pollutant that was produced as a byproduct of combustion. At high concentrations, inhaled nitric oxide is toxic to humans. At low concentrations, researchers have discovered that inhaled nitric oxide can be used to treat various pulmonary diseases in patients. For example, nitric oxide has been investigated for the treatment of patients with increased airway resistance as a result of emphysema, chronic bronchitis, asthma, adult respiratory distress syndrome (ARDS), and chronic obstructive pulmonary disease (COPD).

Nitric oxide has also been investigated for its use as a sterilizing agent. It has been discovered that nitric oxide will interfere with or kill the growth of bacteria grown in vitro, PCT International Application No. PCT/CA99/01123, published Jun. 2, 2000, discloses a method and apparatus for the treatment of respiratory infections by nitric oxide inhalation. Nitric oxide has been found to have either an inhibitory and/or a cidal effect on pathogenic organisms.

While nitric oxide has shown promise with respect to certain medical applications, delivery methods and devices must cope with certain problems inherent with gaseous nitric oxide delivery. For example, exposure to very high concentrations of inhaled nitric oxide is toxic. Even lower levels of inhaled nitric oxide, however, can be harmful if the time of exposure is relatively high. For example, the Occupational Safety and Health Administration (OSHA) has set exposure limits for inhaled nitric oxide in the workplace at 25 ppm time-weighted averaged for eight (8) hours. It is extremely important that any device or system for delivering nitric oxide include features that diminish the leaking of significant amounts of nitric oxide into poorly ventilated spaces of the surrounding environment. If the device is used within a closed space, such as a hospital room or at home, dangerously high levels of nitric oxide can build up in a short period of time.

Another potential problem with using nitric oxide is that nitric oxide oxidizes in the presence of oxygen to form nitric dioxide, which when inhaled is toxic, even at levels lower than those of nitric oxide. If compensatory precautions are not taken, unacceptably high levels of nitric dioxide can develop, especially in closed, unventilated spaces. The rate of oxidation of nitric oxide to nitric dioxide is dependent on numerous factors, including the concentration of nitric oxide, the concentration of oxygen, and the time available for reaction. For example, in a mixture of 1000 ppm nitric oxide and 21% oxygen, it takes about 3.5 minutes for half of the nitric oxide to react to become nitric dioxide (Chironna and Altshuler, 1999, Pollution Engineering p. 33-36). Since nitric oxide will react with the oxygen in the air to convert to nitric dioxide, it is desirable to minimize incidental contact between the nitric oxide gas and the outside environment.

Although Stenzler (see for example, U.S. Pat. Nos. 6,432,077 and 6,793,644 and US Patent App Nos. 2002/0082566, 2002/0156416, and 2005/0137521) and Stenzler et al. (see for example, U.S. Pat. No. 7,122,018 and US Patent App Nos. 2007/0088316, 2005/0191372) describe devices and methods for delivering nitric oxide to treat surface infections and/or wounds, these references warn against allowing gaseous nitric oxide to come into contact with oxygen, because the reaction of nitric oxide and oxygen generates toxic nitric dioxide and consequently leaves less nitric oxide available for treating the surface infection and/or wound. Further, although these references suggest the use of a variety of dilutant gases, including nitrogen, oxygen, and air, for diluting the starting nitric oxide gas to achieve the final desired nitric oxide concentration, none of these references teach specific concentrations of oxygen in a mixture with nitric oxide, or otherwise disclose monitoring the oxygen concentration of the final diluted nitric oxide gas mixture that is delivered.

In addition, Miller et al. (U.S. patent application Ser. No. 11/704,602) describes devices and methods for delivering higher concentrations of nitric oxide for a variety of applications, including the treatment of surface infections and/or wounds. However, Miller does not teach specific concentrations of oxygen in a mixture with nitric oxide, or otherwise disclose monitoring the oxygen concentration of the final diluted nitric oxide gas mixture that is delivered.

There remains a need for a device and method for the treatment of surface and subsurface infections by pathogens, including bacteria, antibiotic resistant bacteria, viruses, protozoa, parasites, arthropods and fungi. The present invention fulfills this need.

### SUMMARY OF THE INVENTION

The present invention provides methods of treating bacterial, viral, protozoan, parasitic, arthropod and fungal infections by applying gaseous nitric oxide to a patient having an infection. The present invention further provides improved methods of treating bacterial, viral, protozoan, parasitic and fungal infections by applying gaseous nitric oxide, in combination with oxygen, to a patient having an infection.

Generally, the invention provides methods of delivering a gas mixture containing an effective amount of nitric oxide, in combination with oxygen, to an infected site of a patient to reduce pathogen levels comprising the steps of: providing a flow-controlled source of a gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; providing a bathing unit around the infected site of the patient, the bathing unit forming a seal with the infected site of the patient; transporting the gas mixture to the bathing unit so as to bathe the infected site of the patient with the gas mixture. In various embodiments, the pathogen is a bacterium, such as an antibiotic-resistant bacteria, a virus, a fungus, a parasite, an arthropod or a protozoan. Non-limiting examples of antibiotic-resistant bacteria include Staphylococcus aureus, Pseudomonas aeruginosa, Streptococcus pneumoniae, Escherichia cell, Salmonella and Klebsiella, Enterococci. The gas mixture can be delivered at 1 atmosphere of pressure or at greater than 1 atmosphere of pressure. In some embodiments, the invention provides methods of delivering a gas containing an effective amount of nitric oxide to an infected site of a patient to reduce pathogen levels comprising the steps of: providing a flow-controlled source of a gas containing nitric oxide, wherein the gas contains at least about 10,000 ppm nitric oxide; providing a bathing unit around the infected site of the patient, the bathing unit forming a seal with the infected site of the patient; transporting the gas to the bathing unit so as to bathe the infected site of the patient with the gas. In various embodiments, the pathogen is a bacterium, such as an antibiotic-resistant bacteria, a virus, a fungus, a parasite, an arthropod or a protozoan. Non-limiting examples of antibiotic-resistant bacteria include Staphylococcus aureus, Pseudomonas aeruginosa, Streptococcus pneumoniae, Escherichia coli, Salmonella and Klebsiella, Enterococci. The gas can be delivered at 1 atmosphere of pressure or at greater than 1 atmosphere of pressure. In some embodiments, the methods include that additional step of agitating the gas within the bathing unit. In various embodiments, the method includes an additional step of stripping nitric oxide from any gas evacuated from the area surrounding the infected site.

In other embodiments, the methods of the invention promote the healing of an area of the body of a patient with a wound or a lesion, the methods comprising the steps of: providing a flow-controlled source of a gas containing at least about 10,000 ppm nitric oxide and delivering the gas to the area of the body so as to bathe the wound with the gas containing nitric oxide. and wherein the wound is infected by at least one type of pathogen. Optionally, the gas containing nitric oxide also contains at least about 20% oxygen, and the method additionally includes the steps of providing a flow-controlled source of gas containing oxygen and mixing the nitric oxide with the oxygen before delivering the gas mixture to the wound. In various embodiments, the wound or lesion is, for example, a surgical wound, a trauma would, a burn or an abscess. In various embodiments, the pathogen is a bacterium, such as an antibiotic-resistant bacteria, a virus, a fungus, a parasite, an arthropod or a protozoan. Non-limiting examples of antibiotic-resistant bacteria include Staphylococcus aureus, Pseudomonas aeruginosa, Streptococcus pneumoniae, Escherichia coli, Salmonella and Klebsiella, Enterococci. The gas can be delivered at 1 atmosphere of pressure or at greater than 1 atmosphere of pressure. In some embodiments, the methods include that additional step of agitating the gas near the wound or lesion. In various embodiments, the method includes an additional step of stripping nitric oxide from any gas evacuated from the area of the body of a patient with a wound or a lesion.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts a schematic representation of the nitric oxide delivery device according to one aspect of the invention.

FIG. 2 depicts a bathing unit surrounding the foot of a patient.

FIG. 3 depicts a bathing unit surrounding the hand of a patient.

FIG. 4 depicts a bathing unit including an agitator located therein.

FIG. 5 depicts an image of a local wound cover bathing unit that uses just a single tube to flow the gas into the bathing unit and allows the gas to exit through one or more holes in the top occlusive cover.

FIG. 6 depicts an image of a bathing unit that uses a single tube to flow the gas into the bathing unit, the top portion of which is raised above the treatment surface.

FIG. 7 depicts a drawing indicating arrangement of wounds and treatment parameters used in Experimental Example 1.

FIG. 8 depicts a graph of the results of an example experiment (see Experimental Example 1) demonstrating, in part, that treatment with 10,000 ppm nitric oxide in combination with 20% oxygen diminishes bacterial counts in infected wounds more than 10,000 ppm nitric oxide in combination with nitrogen (i.e. without added oxygen).

FIG. 9 depicts a graph of the results of an example experiment (see Experimental Example 1) demonstrating, in part, that treatment with 10,000 ppm nitric oxide in combination with 20% oxygen diminishes bacterial counts in infected wounds more than 10,000 ppm nitric oxide in combination with nitrogen (i.e. without added oxygen).

FIG. 10 depicts a graph of the results of an example experiment (see Experimental Example 1) demonstrating, in part, that treatment with 10,000 ppm nitric oxide in combination with 20% oxygen diminishes bacterial counts in infected wounds more than 10,000 ppm nitric oxide in combination with nitrogen (i.e. without added oxygen).

FIG. 11 depicts a drawing indicating arrangement of wounds and treatment parameters used in Experimental Example 2.

FIG. 12 depicts a graph of the results of an example experiment (see Experimental Example 2) demonstrating, in part, that treatment with 10,000 ppm nitric oxide in combination with 20% oxygen diminishes bacterial counts in infected wounds more than treatment with 10,000 ppm nitric oxide in combination with 15% oxygen, which in turn diminishes bacterial counts in infected wounds more than treatment with 10,000 ppm nitric oxide in combination with 10% oxygen.

FIG. 13 depicts a graph of the results of an example experiment (see Experimental Example 2) demonstrating, in part, that treatment with 10,000 ppm nitric oxide in combination with 20% oxygen diminishes bacterial counts in infected wounds more than treatment with 10,000 ppm nitric oxide in combination with 15% oxygen, which in turn is diminishes bacterial counts in infected wounds more than treatment with 10,000 ppm nitric oxide in combination with 10% oxygen.

FIG. 14 depicts a graph of the results of an example trial evaluating the effectiveness of gaseous nitric oxide treatment against mild tinea pedis in human patients.

FIG. 15 depicts the results of an example trial evaluating the effectiveness of gaseous nitric oxide treatment against moderate-severe interdigital and/or moccasin-type tinea pedis in human patients.

FIG. 16 depicts the results of an example trial evaluating the effectiveness of gaseous nitric oxide treatment against moderate-severe interdigital and/or moccasin-type tinea pedis in human patients.

FIG. 17 depicts the results of an example trial evaluating the effectiveness of gaseous nitric oxide treatment against moderate-severe interdigital and/or moccasin-type tinea pedis in human patients.

FIG. 18 depicts the results of an example trial evaluating the effectiveness of gaseous nitric oxide treatment against moderate-severe interdigital and/or moccasin-type tinea pedis in human patients.

FIG. 19 depicts the an example device for the delivery of nitric oxide containing gas to the foot of a patient with tinea pedis.

FIG. 20 depicts the results of an example experiment evaluating the effectiveness of gaseous nitric oxide against lice and lice eggs.

### DETAILED DESCRIPTION

It has been unexpectedly discovered in the present invention, that delivering a mixture of nitric oxide and oxygen provides enhanced anti-infective properties over the delivery of nitric oxide without oxygen. Further, it has been unexpectedly discovered in the present invention, that delivering a mixture of nitric oxide and near-atmospheric concentrations of oxygen provides enhanced anti-infective properties over the delivery of nitric oxide mixed with oxygen at concentrations substantially lower than the concentration of oxygen found in air (i.e., about 21%). Thus, the present invention provides an improved method of treating bacterial, viral, protozoan, parasitic and fungal infections by applying gaseous nitric oxide, in combination with oxygen, to a patient having an infection. In one aspect, the invention provides a method of treating a bacterial infection, such as, for example, an antibiotic-resistant bacterial infection, by applying gaseous nitric oxide, in combination with oxygen, to a site of infection of a patient. In another aspect, the invention provides a method of treating a fungal infection by applying gaseous nitric oxide, in combination with oxygen, to a site of infection of a patient. In still another aspect, the invention provides a method of treating a viral infection by applying gaseous nitric oxide, in combination with oxygen, to a site of infection of a patient. In a further aspect, the invention provides a method of treating a parasitic infection by applying gaseous nitric oxide, in combination with oxygen, to a site of infection of a patient. In a still further aspect, the invention provides a method of treating a protozoan infection by applying gaseous nitric oxide, in combination with oxygen, to a site of infection of a patient.

The delivery of nitric oxide in combination with near-atmospheric concentrations of oxygen represents an improvement over existing devices and methods that deliver nitric oxide without oxygen, as well as those that deliver nitric oxide in combination with oxygen at concentrations substantially lower than the concentration of oxygen found in air. As detailed herein, the delivery of nitric oxide, in combination with oxygen, is able to kill a higher fraction of pathogens than the delivery of nitric oxide in the absence of oxygen. Although in various embodiments of the invention described herein, the delivery of nitric oxide, in combination with oxygen, exhibits enhanced anti-infective properties over the delivery of nitric oxide mixed with oxygen at concentrations substantially lower than the concentration of oxygen found in air, the skilled artisan will understand that the invention described herein also includes nitric oxide, in combination with oxygen at concentrations substantially lower than the concentration of oxygen found in air, including mixtures that contain little or no oxygen.

The present invention specifically provides a method of treating various infections, including those caused by viruses, fungi, protozoans, parasites, arthropods and bacteria, including bacteria that have developed resistance to one or more antibiotics. Examples of some bacteria known to have developed resistance to one or more antibiotics include, Staphylococcus aureus, Pseudomonas aeruginosa, Streptococcus pneumoniae, Escherichia coli, Salmonella, Klebsiella, and Enterococci. Typically, gaseous nitric oxide, in combination with oxygen, is administered to the site of infection of a patient using a device for delivery of the gas mixture to the site of infection. The device preferably minimizes leaking to avoid the potentially dangerous build up of nitric oxide and nitric dioxide concentrations in the surrounding environment. The application of nitric oxide to the affected region preferably decreases the time required to heal the affected area. Although not wishing to be bound by any particular theory, improved healing by the application of nitric oxide can occur by the reduction of the level of the pathogen, by the improvement of blood supply to the affected tissues through vasodilation, by the reduction of inflammation, or by a combination thereof. Especially for deliveries of higher concentrations of nitric oxide, for example greater than 1000 ppm, the device optionally includes a nitric oxide and/or nitric dioxide absorber or scrubber that will remove or chemically alter nitric oxide and/or nitric dioxide prior to or during discharge from the delivery device.

In an aspect of the invention, the application of nitric oxide, in combination with oxygen, to the affected region decreases the time required to heal the affected area, induces healing of the affected area, or directly contributes to the healing of the area. In an embodiment of the invention, application of nitric oxide, in combination with oxygen, to the affected region acts by two or more different mechanisms to promote healing.

In an aspect of the invention, the improvement of symptoms in a patient being treated with a composition of the invention, a method of the invention, or both, and/or the promotion of healing, can be measured by any one of many ways known in the art. Generally, promotion of healing can be ascertained by the artisan skilled in treating the type of infection being treated.

While the methods of the invention should not be construed to be limited solely to the mechanisms and devices described herein for delivery of nitric oxide, in combination with oxygen, to a site of infection, certain embodiments encompassing various mechanisms and devices are described herein as examples of how to perform the methods of the invention. It is understood that the methods of the invention may be practiced using other known or heretofore unknown mechanisms and devices and the invention should be construed to encompass all such mechanisms and devices as appropriate.

In a first aspect of the invention, a device for the delivery of nitric oxide gas, to a site of infection includes a source of nitric oxide gas, an optional source of oxygen gas, a bathing unit, and a flow control valve, Optionally, the device for the delivery of nitric oxide gas may also contain an absorber, and/or a scrubber, and/or a vacuum unit. The bathing unit is in fluid communication with the source of nitric oxide gas and with the optional source of oxygen gas and is adapted for surrounding the site of infection. The flow control valve is positioned downstream of the source of gases and upstream of the bathing unit for controlling the amount of nitric oxide gas mixture that is delivered to the bathing unit. The optional vacuum unit may be positioned downstream of the bathing unit for withdrawing gas from the bathing unit.

In a second aspect of the invention, the device according to the first aspect of the invention includes a controller for controlling the operation of the flow control valve and an optional vacuum unit.

In a third aspect of the invention, the device according to the first aspect of the invention further includes a gas blender. The nitric oxide gas and the optional oxygen gas are mixed by the gas blender. The device also optionally includes a nitric oxide gas absorber or scrubber unit that is positioned upstream of the vacuum unit. The device further includes a controller for controlling the operation of the flow control valve and the vacuum unit.

In a fourth aspect of the invention, the device according to the first aspect of the invention further includes a source of a third dilutent gas. The third dilutent gas and the nitric oxide gas and the optional oxygen gas can be mixed together using a gas blender.

In a fifth aspect of the invention, a method of delivering an effective amount of nitric oxide to a site of infection includes the steps of providing a bathing unit around the site of infection. Gas containing the nitric oxide mixture is then transported to the bathing unit so as to bathe the site of infection with gaseous nitric oxide and optional oxygen. Optionally, at least a portion of the delivered gas is evacuated or diffuses from the bathing unit.

A delivery device for the delivery of a nitric oxide gas mixture to a site of infection is thus provided. The device is preferably constructed to limit the amount of nitric oxide-containing gas leaking from the delivery device. The method of delivering an effective amount of nitric oxide gas mixture to the site of infection kills the pathogen and promotes the healing process.

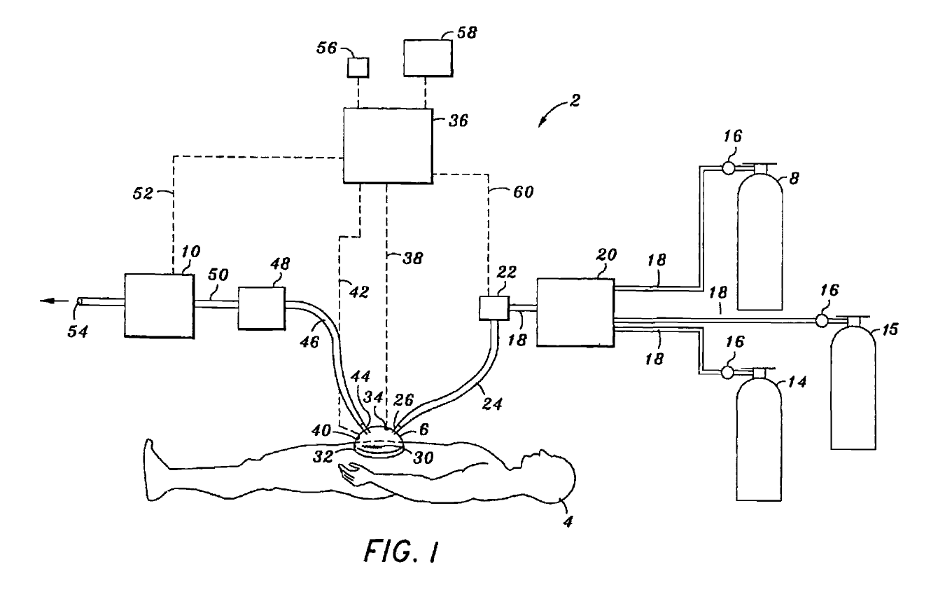

In one set of embodiments and referring now to FIG. 1 herein, a nitric oxide delivery device 2 is shown connected to a patient 4. In its most general sense, the nitric oxide delivery device 2 includes a bathing unit 6 that is fluidically connected to a nitric oxide gas source 8, an optional oxygen gas source 15, a flow control valve 22, and an optional vacuum unit 10. FIG. 1 therefore illustrates one preferred embodiment of the device useful for administration of a nitric oxide gas mixture to a patient in the methods of the invention, Although FIG. 1 depicts a bathing unit 6 in contact with the surface of the mid-region of a patient, it should be understood that the bathing unit 6 can be modified as appropriate to bathe any site of infection on the surface of, or inside the body of, a patient.

In FIG. 1, the nitric oxide gas source 8 is a pressurized cylinder containing nitric oxide gas. While the use of a pressurized cylinder is the preferable method of storing the nitric oxide-containing gas source 8, other storage and delivery means, such as a dedicated feed line (wall supply) or a nitric oxide generator can also be used. Typically, the nitric oxide gas source 8 is a mixture of nitrogen and nitric oxide. While nitrogen is typically used to dilute the concentration of nitric oxide within the pressurized cylinder, any inert gas can also be used. When the nitric oxide gas source 8 is stored in a pressurized cylinder, it is preferable that the concentration of nitric oxide in the pressurized cylinder fall within the range of about 1000 ppm to about 200,000 ppm or higher. In certain settings, extremely high concentrations of nitric oxide may be undesirable because unintentional leakage of nitric oxide gas may be more hazardous. Pressurized cylinders containing low concentrations of nitric oxide (i.e., less than 1000 ppm nitric oxide) can also be used in accordance the device and method disclosed herein. Of course, the lower the concentration of nitric oxide used, the more often the pressurized cylinders will need replacement.

In FIG. 1, the optional oxygen gas source 15 is a pressurized cylinder containing oxygen gas. While the use of a pressurized cylinder is the preferable method of storing the oxygen-containing gas source 15, other storage and delivery means, such as a dedicated feed line (wall supply), an oxygen concentrator or an oxygen generator, can also be used. Alternatively, room air can be entrained into the flow of the gas mixture as the source of oxygen. Typically, the oxygen gas source 15 is nearly pure oxygen, but could instead be a mixture of oxygen and an inert gas. While nitrogen is typically used to dilute the concentration of oxygen within the pressurized cylinder, any inert gas can also be used.

FIG. 1 also shows the source of an optional dilutant gas 14 as part of the nitric oxide delivery device 2 that is used to dilute the concentration of nitric oxide. The source of dilutent gas 14 can contain either nitrogen, air, an inert gas, or a mixture of these gases. The source of dilutent gas 14 is shown as being stored within a pressurized cylinder. While the use of a pressurized cylinder is shown in FIG. 1 as the means for storing the source of dilutent gas 14, other storage and delivery means, such as a dedicated feed line (wall supply), or air pump, can also be used.

It should be understood that, although FIG. 1 depicts a system having three sources of gas (i.e., nitric oxide gas source 8, an optional oxygen gas source 15, and the optional dilutent gas source 14), the concentrations and ratios of nitric oxide and oxygen in the gas mixtures described herein below can also be accomplished by using a system having only two sources of gas (i.e., nitric oxide gas source 8 and oxygen gas source 15), depending on the initial concentration of the nitric oxide in gas source 8 and the oxygen concentration in gas source 15.

The nitric oxide gas from the nitric oxide gas source 8, and the oxygen gas from the optional oxygen gas source 15, and the optional dilutent gas from the dilutent gas source 14 preferably pass through pressure regulators 16 to adjust the pressure of gas that is admitted to the gas delivery device 2 to the appropriated desired pressure and the appropriate desired concentration. The respective gas streams pass via tubing 18 to an optional gas blender 20. The gas blender 20 mixes the nitric oxide gas, the oxygen gas, and the optional dilutent gas to produce a nitric oxide gas mixture that has the desired concentrations of nitric oxide and, optionally, oxygen. In various embodiments, the gas mixture that is output from the gas blender 20 has a concentration of nitric oxide that ranges from about 1000 ppm to about 40,000 ppm. In various embodiments, the gas mixture that is output from the gas blender 20 has a concentration of nitric oxide that ranges from about 1000 to about 5000 ppm, from about 4000 to about 10,000 ppm, from about 9,000 to about 16,000 ppm, from about 15,000 to about 22,000 ppm, from about 21,000 to about 28,000 ppm, from about 27,000 to about 34,000 ppm, and from about 33,000 to about 40,000 ppm. Preferably, the gas mixture that is output from the gas blender 20 has a concentration of nitric oxide that is about 30,000 ppm. Even more preferably, the concentration of nitric oxide in the gas mixture that is output from the gas blender 20 is about 20,000 ppm nitric oxide. Even more preferably, the concentration of nitric oxide in the gas mixture that is output from the gas blender 20 is about 10,000 ppm nitric oxide. In various embodiments, the gas mixture that is output from the gas blender 20 has a concentration of oxygen that ranges from about 16% to about 30%. In various embodiments, the gas mixture that is output from the gas blender 20 has a concentration of oxygen that ranges from about 16 to about 20%, from about 18 to about 22%, from about 20 to about 24%, from about 22 to about 26%, from about 24 to about 28%, and from about 26 to about 30%. Preferably, the gas mixture that is output from the gas blender 20 has a concentration of oxygen that is at least about 16%. Even more preferably, the concentration of oxygen in the gas mixture that is output from the gas blender 20 is at least about 18%. Even more preferably, the concentration of oxygen in the gas mixture that is output from the gas blender 20 is at least about 20%.

In an aspect, the nitric oxide concentration in the gas mixture used for treatment can fall in the range of 1000 ppm to 40,000 ppm. For a given application, the concentration of nitric oxide selected, as well as the exposure time selected, will vary according to a variety of circumstances including, for example, the particular site of infection. By way of a non-limiting example, a higher concentration of nitric oxide may be used when a shorter treatment time is desired. By way of other non-limiting examples, a high concentration of nitric oxide could be applied for a relatively short time (e.g., 10,000 ppm for 30 minutes or 30,000 ppm for 20 minutes). By way of another non-limiting example, a low concentration of nitric oxide could be applied for a relatively long time (e.g., 1000 ppm for 8 hours). Moreover, a particular site of infection may be treated only once, or a particular site of infection may be treated repeatedly over a period of days or weeks. Based on the disclosure set forth herein, the skilled artisan will understand how to adjust the concentration of nitric oxide, and moreover, how to select the concentration of nitric oxide necessary for any particular application. However, it will be understood that the present application also teaches the skilled artisan how to determine the concentration of nitric oxide, and the concentration of oxygen, useful for any particular set of circumstances, based on the time of treatment and also based on the desired outcome of the treatment (e.g., alleviation of symptoms versus eradication of the infection).

The gas mixture containing nitric oxide and oxygen that is output from the gas blender 20 travels via tubing 18 to a flow control valve 22. The flow control valve 22 can include, for example, a proportional control valve that opens (or closes) in a progressively increasing (or decreasing if closing) manner. As another example, the flow control valve 22 can include a mass flow controller. The flow control valve 22 controls the flow rate of the gas mixture that is input to the bathing unit 6. The gas mixture leaves the flow control valve 22 via flexible tubing 24. The flexible tubing 24 attaches to an inlet 26 in the bathing unit 6. The inlet 26 might include an optional one way valve 64 (see FIG. 3) that prevents the backflow of the gas mixture into the tubing 24.

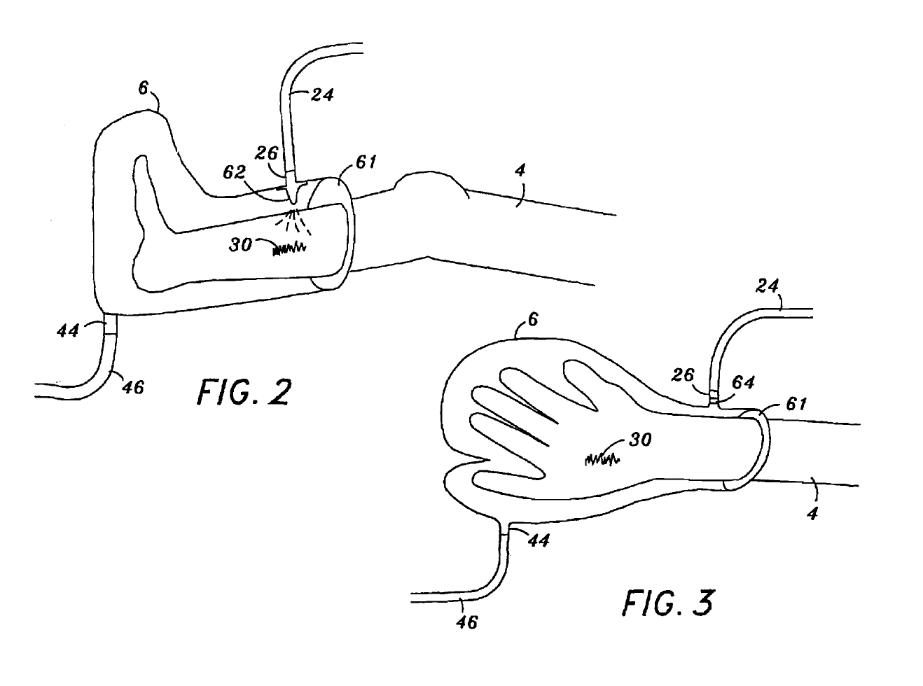

Still referring to FIG. 1, the bathing unit 6 is shown connected to the surface of a patient 4. Although FIG. 1 depicts a bathing unit 6 connected to the surface of the mid-region of a patient 4, it should be understood that the bathing unit 6 can be modified as appropriate to bathe any site of infection on the surface of, or inside the body of, a patient. The infected area 30 which can be an abscess, lesion, wound, surgical site, apparently intact tissue, burn, or the like, is enclosed by the bathing unit 6. The bathing unit 6 optionally includes a seal portion 32, which can be used to form a seal with patient 4. In some embodiments, the optional seal portion 32 can be used to form a substantially air-tight seal to diminish the amount of nitric oxide-containing gas that leaks out of the bathing unit 6 (e.g., no more than about 5% of the nitric oxide-containing gas delivered to the bathing unit 6). In other embodiments, the optional seal portion 32 forms a seal that is not an air-tight seal and nitric oxide-containing gas can leak out of the bathing unit. The seal portion 32 may is comprise an inflatable seal 61, such as that shown in FIGS. 2 and 3, or alternatively the seal portion 32 may comprise a flexible skirt or the like that conforms to the surface of the patient 4. The seal portion 32 also might include an adhesive portion that adheres to the surface of a patient 4. In other various embodiments, the seal portion 32 may merely comprise the interface of the bathing unit 6 with the patient 4.

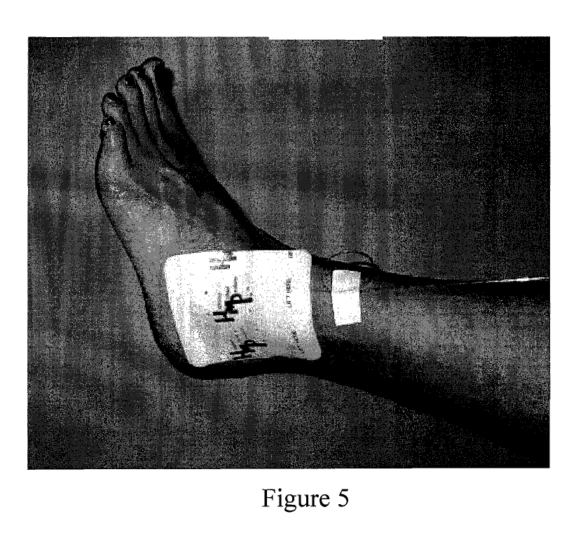

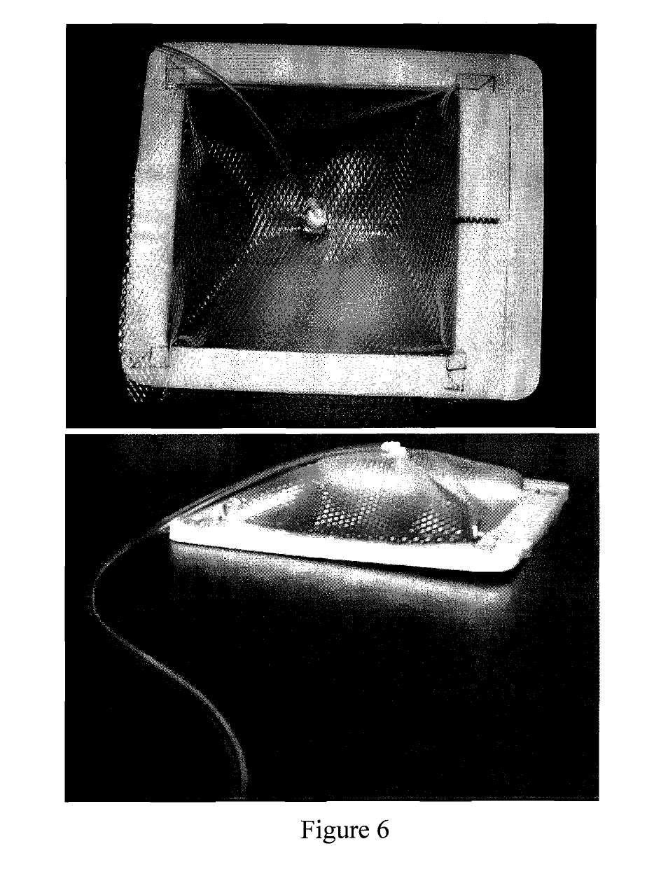

The bathing unit 6 can be made of a virtually limitless number of shapes and materials depending on its intended use. The bathing unit 6 might be formed as a rigid structure, such as that depicted in FIG. 1, that is placed over the infected area 30. Alternatively, the bathing unit 6 can be formed of a flexible, bag-like material that is inflatable over the infected area 30. FIG. 2 shows such a structure in the shape of a boot that is placed over the patient's 4 foot, FIG. 3 shows another inflatable bathing unit 6 that is formed in the shape of a mitten or glove that is worn over the patient's 4 hand. FIG. 5 depicts an image of an example local wound cover bathing unit that uses a single tube to flow the gas into the bathing unit and allows the gas to exit through one or more holes in the top occlusive cover. FIG. 6 depicts an image of an example bathing unit that uses a single tube to flow the gas into the bathing unit, the top portion of which is raised above the treatment surface.

In a preferred embodiment of the invention, the bathing unit 6 includes an nitric oxide sensor 34 that measures the concentration of nitric oxide gas within the bathing unit 6. The nitric oxide sensor 34 preferably reports this information to a controller 36 via signal line 38. An optional nitric dioxide sensor 40 can also be included within the bathing unit 6. The nitric dioxide sensor 40 preferably reports the concentration of nitric dioxide to the controller 36 via signal line 42. The sensors 40, 42 can be a chemilluminesence-type, electrochemical cell-type, or spectrophotometric-type sensor.

The bathing unit 6 also includes an outlet 44 that can be used to allow the gas mixture to exit the bathing unit 6. The outlet 44 is preferably located away from the gas inlet 26 such that gas mixture does not quickly enter and exit the bathing unit 6. Preferably, the inlet 26 and outlet 44 are located in areas of the bathing unit 6 such that the gas mixture has a relatively long residence time. Flexible tubing 46 is connected to the outlet 44 and provides a conduit for the removal of gases from the bathing unit 6.

In another preferred embodiment of the invention, the flexible tubing 46 is in fluid communication with an absorber unit 48. The absorber unit 48 preferably absorbs, scrubs, or strips nitric oxide from the gas stream that is exhausted from the bathing unit 6. It is also preferable for the absorber unit 48 to also absorb, scrub, or strip nitric dioxide from the gas stream that is exhausted from the bathing unit 6. Since these gases are toxic at high levels when inhaled, it is preferable that these components are removed from the delivery device 2 prior to the gas being vented to the atmosphere. In addition, these gases can react with the internal components of the optional vacuum unit 10 and potentially interfere with the operation of the delivery device 2.

The now clean gas travels from the absorbing unit 48 to an optional vacuum unit 10 via tubing 50. The optional vacuum unit 10 provides a negative pressure within the tubing 50 so as to extract gases from the bathing unit 6. The optional vacuum unit 10 is preferably controllable with respect to the level of vacuum or suction supplied to the tubing 50 and bathing unit 6. In this regard, in conjunction with the flow control valve 22, the amount of nitric oxide gas within the bathing unit 6 can be regulated. Preferably, the vacuum unit 10 is coupled with the controller 36 via a signal line 52. The controller 36, as discussed below, preferably controls the level of output of the vacuum unit 10. The gas then passes from the vacuum unit 10 to a vent 54 that is open to the atmosphere.

It should be understood that the vacuum unit 10, as well as the absorbing unit 48, are optional components of the delivery device 2. The gas laden with nitric oxide and nitric dioxide does not have to be removed from the gas stream if there is no concern with local levels of nitric oxide and nitric dioxide. For example, the gas can be exhausted to the outside environment where high concentrations of nitric oxide and nitric dioxide will not develop. Alternatively, a recirculation system (not depicted in FIG. 1) might be used to recycle the gas mixture or the nitric oxide within the bathing unit 6.

Still referring to FIG. 1, the delivery device 2 preferably includes a controller 36 that is capable of controlling the flow control valve 22 and the vacuum unit 10. The controller 36 is preferably a microprocessor-based controller 36 that is connected to an input device 56. The input device 56 is used by an operator to adjust various parameters of the delivery device such as nitric oxide concentration in the mixture, residence time of nitric oxide, pressure within the bathing unit 6, etc. An optional display 58 can also be connected with the controller 36 to display measured parameters and settings such as the set-point nitric oxide concentration, the concentration of nitric oxide within the bathing unit 6, the concentration of nitric dioxide within the bathing unit 6, the flow rate of gas into the bathing unit 6, the flow rate of gas out of the bathing unit 6, the total time of delivery, and the like.

The controller 36 preferably receives signals from sensors 34, 40 regarding gas concentrations if such sensors 34, 40 are present within the delivery device 2. Signal lines 60, 52 are connected to the flow control valve 22 and vacuum unit 10 respectively for the delivery and receipt of control signals.

In another embodiment of the invention, the controller 36 is eliminated entirely. In this regard, the flow rate of the gas into the bathing unit 6 and the flow rate of the gas out of the bathing unit 6 are pre-set or adjusted manually. For example, an operator can set a vacuum output that is substantially equal to the flow rate of the gas delivered to the bathing unit 6 via the flow control valve 22. In this manner, nitric oxide gas will be able to bathe the infected area 30 without any build-up or leaking of nitric oxide or nitric dioxide gas from the delivery device 2.

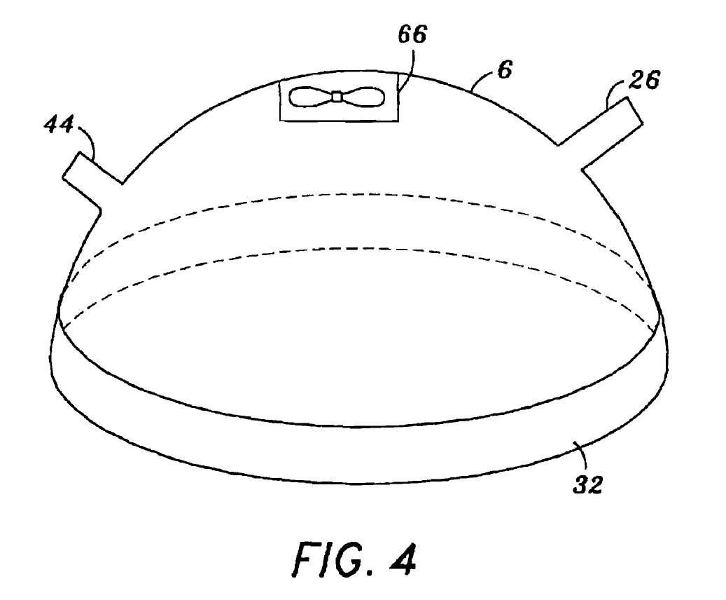

FIG. 2 illustrates a bathing unit 6 in the shape of a boot that is used to treat an infected area 30 located on the leg of the patient 4. The bathing unit 6 includes an inflatable seal 61 that surrounds the leg region to make a seal with the surface of the patient 4, This embodiment shows a nozzle 62 that is affixed near the inlet 26 of the bathing unit 6. The nozzle 62 directs a jet of nitric oxide gas, in combination with oxygen, onto the infected area 30, The jet of gaseous nitric oxide aids in penetrating the infected area 30 with nitric oxide to kill or inhibit the growth of pathogens. FIG. 3 shows another embodiment of the bathing unit 6 in the shape of a mitten or glove. The bathing unit 6 is also inflatable and contains an inflatable seal 61 that forms a seal around the surface of the patient 4. FIG. 3 also shows an optional one way valve 64 located in the inlet 26. As seen in FIGS. 3 and 4, the inlet 26 and outlet 44 are located away from one another, and preferably on opposing sides of the treated area such that freshly delivered nitric oxide gas is not prematurely withdrawn from the bathing unit 6.

For treatment of an infected area 30, the bathing unit 6 is placed over the infected area 30. A seal is then formed between the surface of the patient 4 and the bathing unit 6. If the bathing unit 6 has an inflatable construction, the bathing unit 6 must be inflated with gas. Preferably, the bathing unit 6 is initially inflated only with the dilutent gas to prevent the leaking of nitric oxide and nitric dioxide from the device 2. Once an adequate seal has been established, the operator of the device initiates the flow of nitric oxide from the nitric oxide gas source 8 to the bathing unit 6. As described above, this may be accomplished manually or via the controller 36. The skilled artisan will know how to establish the appropriate seal—either air-tight, or less than air-tight (e.g., “free-flowing”)—depending upon the particular objective and goal for treatment under any particular set of facts.

Once the bathing unit 6 has started to fill with nitric oxide gas, the optional vacuum unit 10 can be turned on and adjusted to the appropriate output level. For an inflatable bathing unit 6, the output level (i.e., flow rate) of the vacuum unit 10 should be less than or equal to the flow rate of nitric oxide gas entering the bathing unit 6 to avoid deflating the bathing unit 6 and/or to allow the pressure in the bathing unit to increase. In some embodiments, the vacuum unit 10 can be set to withdraw gas at a substantially equal rate as the gas is delivered to the bathing unit 6. An effective amount of nitric oxide, in combination with oxygen, is delivered to the bathing unit 6 to kill pathogens and/or reduce the growth rate of the pathogens in the infected area 30. Pathogens include antibiotic-resistant bacteria, other bacteria, viruses, protozoa, parasites, arthropods and fungi.

FIG. 4 shows another embodiment of the invention in which the bathing unit 6 includes an agitator 66 that is used to create turbulent conditions inside the bathing unit 6. The agitator 66 preferably is a fan-type of mechanism but can include other means of creating turbulent conditions within the bathing unit 6. The agitator 66 aids in refreshing the affected area 30 with a fresh supply of gas containing nitric oxide and oxygen.

FIG. 5 illustrates another embodiment of the invention, in which the bathing unit comprises a wound cover that has one or more openings for the release of nitric oxide gas. In an embodiment, nitric oxide gas is introduced underneath the bathing unit, such that the gaseous nitric oxide is largely contained beneath the unit, in a space between the unit and the surface of the patient. However, the nitric oxide gas and spent nitric oxide gas are free to escape through the one or more openings in the bathing unit. FIG. 6 illustrates another embodiment of the invention, in which the bathing unit comprises a wound cover, the top is portion of which is raised above the treatment surface.

As will be understood by the skilled artisan, when armed with the present disclosure, variations in the number and/or size of the openings in such a bathing unit can be used to regulate the pressure of nitric oxide gas underneath the wound cover, and/or to regulate the amount of time that the gaseous nitric oxide is in contact with the a site of infection beneath the wound cover. It will also be understood that in any method or apparatus of the invention, the nitric oxide gas that is delivered to a patient may optionally be evacuated after use. The skilled artisan will understand, when armed with the disclosure set forth herein, how to determine the timing and method of evacuation.

In one embodiment of the invention, gaseous nitric oxide is applied to the site of infection with a pressure of about 1 atmosphere. In other embodiments, gaseous nitric oxide is applied to the site of infection at a pressure great than 1 atmosphere, such as for example, 1.5, 2, 2.5, 3, 3.5 or 4 or more atmospheres. Without wishing to be bound to any particular theory, the delivery of gaseous nitric oxide to a site of infection at a higher than ambient pressure can serve to increase the penetration of the nitric oxide deeper into the site of infection and/or surrounding tissues. For embodiments delivering gaseous nitric oxide at pressures near about 1 atmosphere, the devices described herein can contain a substantially air-tight seal that allows for a build-up of pressure near about 1 atmosphere. For embodiments delivering gaseous nitric oxide at pressures greater than about 1 atmosphere, the devices described herein can contain a substantially air-tight seal that allows for a build-up of pressure to greater than about 1 atmosphere. Such embodiments can be further modified to have a regulator or valve, such as for example a pop-off valve, on the effluent path that can allow for the selection of a pressure near about 1 atmosphere or greater than about 1 atmosphere. For embodiments delivering gaseous nitric oxide at pressures substantially greater than 1 atmosphere, for example 3 atmospheres, a hyperbaric chamber so can be utilized to surround the site of infection, and/or the entire individual.

Throughout this disclosure, various aspects of this invention can be presented in a range format. It should be understood that the description in range format is merely for convenience and brevity and should not be construed as an inflexible limitation on the scope of the invention, Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.3, 3, 4, 5, 5.7 and 6. This applies regardless of the breadth of the range.

### Definitions:

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “modulate” is meant to refer to any change in biological state, i.e. increasing, decreasing, and the like.

As used herein, a “therapeutically effective amount” is an amount of a therapeutic composition sufficient to provide a beneficial effect to a mammal to which the composition is administered.

The terms “patient,” “subject” and “individual” are interchangeably used to mean a warm-blooded animal, such as a mammal, suffering from a disease, such as, but not limited to, a fungal, a parasitic or an antibiotic-resistant bacterial infection. It is understood that humans and animals are included within the scope of the term “patient,” “subject” or “individual.”

As used herein, the terms “infected site” and “site of infection” are used to mean an area, a region or a site on the surface of, or inside the body of, a patient that exhibits a wound, a lesion, an abscess or other signs or symptoms of infection or colonization, including intact skin. The infected areas, regions and sites that can be treated by the methods of the invention include any area, region or site on the surface of, or inside the body of, a patient that can be exposed to gaseous nitric oxide, in combination with oxygen. By way of is nonlimiting examples, infected areas, regions and sites that can be treated by the methods of the invention include, but are not limited to, external tissues (e.g. skin, etc.), internal tissues (e.g. mucosa, muscle, fascia, etc.), and internal organs (e.g. lungs, liver, etc.). It should be understood that many areas, regions and sites that are normally not amenable to exposure to gaseous nitric oxide, in combination with oxygen, can become amenable to exposure to gaseous nitric oxide, in combination with oxygen, after a wound, such as, for example, a surgical incision or traumatic laceration, is introduced to the body of a patient. Moreover, “infected site” should not be construed to include only those areas, regions or sites that exhibit overt evidence of infection, but rather should also be construed to include areas, regions or sites that may be colonized, subclinically infected and/or asymptomatic, i.e., that do not contain overt evidence of infection, but that may be affected nonetheless and that could, in time, exhibit more overt evidence of infection. By way of nonlimiting examples, such a site can include a trauma wound, surgical wound, intact tissue or burn that has come into contact with, or which is at risk of potentially coming into contact with, a pathogen that can colonize or infect the wound, and can be treated, or prophylactically treated, with the devices and methods of the invention.

The term “treat” or “treatment,” as used herein, refers to the alleviation (i.e., “diminution”) and/or the elimination of a symptom or a source of a symptom or of a given disease. By way of several non-limiting examples, a symptom of a bacterial infection can be treated by alleviating a symptom of that disorder. A symptom of a bacterial infection can also s be treated by altogether eliminating a symptom of that disorder. A bacterial infection or colonization can be treated by alleviating the source, or “cause,” of that disorder. A bacterial infection or colonization can also be treated by eliminating the source of that disorder.

“Evacuating” as the term is used herein, refers to the partial or complete removal of a substance from a specific region or area, This removal can be accomplished actively (e.g., vacuum or displacement) or passively (e.g., diffusion). By way of two separate non-limiting examples according to the present invention, nitric oxide gas can be partially removed from the area under an enclosed device which surrounds the site of infection of a patient, or nitric oxide gas can be completely removed from the area under an enclosed device which surrounds a portion of the a site of infection of a patient. In these examples, the is nitric oxide gas may or may not be replaced with a “substitute” gas. The “substitute” gas, according to the invention, may be any gas, including nitric oxide gas.

As used here, the term “pathogen” refers to an infectious and/or parasitic organism, including, but not limited to, viruses, fungi, protozoa, parasites, arthropods, helminthes, and bacteria, including antibiotic-resistant bacteria. Examples of fungi include, but are not limited to, Epidermophyton floccosum, Epidermophyton sp., Microsporum canis, Microsporum gypseum, Microsporum sp., Trichophyton mentagrophytes, Trichophyton rubrum, Trichophyton tonsurans, Trichophyton verrucosum, Trichophyton sp., Exserohilum sp., Exophiala sp., Candida albicans, Candida glabrata, Candida guilliermondii, Candida krusei, Candida lusitaniae, Candida parapsilosis, Candida tropicalis, Candida sp., Cryptococcus gattii, Cyptococcus neoformans, Cyptococcus sp., Geotrichum sp., Malassezia sp., Pneumocystis jiroveci, Rhodotorula sp., Saccharomyces sp., Trichosporon sp., Blastomyces dermalitidis, Coccidioides immitis, Coccidioides posadasii, Coccidioides sp., Histoplasma capsulatum, Paracoccidioides brasiliensis, Penicillium marneffei, Sporothrix schenckii, Acremonium sp., Aspergillus flavus, Aspergillus fumigates, Aspergillus niger, Aspergillus sp., Alternaria sp., Aureobasidium sp., Bipolaris sp., Cladophialophora sp., Cladosporium sp., Fonsecaea sp., Hortaea werneckii, Madurella sp., Phialophora sp., Piedraia horlae, Wangiella dermatitidis, Absidia sp., Cunninghamella, Mucor sp., Rhizomucor sp., Rhizopus sp. Examples of protozoa include, but are not limited to, Acanthamoeba castellanii, Acanthamoeba species, Balamuthia mandrillaris, Endolimax nana, Entamoeba dispar, Entamoeba histolytica, Entamoeba moshkovskii, Entamoeba specks, Hartmanella species, Lodamoeba butschlii, Naegleria fowleri, Naegleria species, Balantidium coli, Chilomastix memili, Dientamoeba fragi ils, Enteromonas hominis, Giardia Limblia, Leishsmania braziliensis complex, Leishmania donovani complex, Leishmania major, Leishmania mexicana complex, Leishmania tropica, Leishmania species, Retortamonas inCestinalis, Trichomonas vaginalis, Trichomonas species, Trypanosoma brucei gambiense, Trypanosoma brucei rhodesiense, Typanosoma cruzi, Babesia species, Cryptosporidium hominis, Cryptosporidium parvum, Cyclospora cayetanensis, Isospora belli, Plasmodium falciparum, Plasmodium malariae, Plasmodium ovale, Plasmodium vivax, Sarcocystis hominis, Sarcocystis species, Toxoplasma gondii, Bracheola species, Encephalitozoon helium, Encephalitozoon intestinalis, Encephalitozoon species, Enterocytozoon bieneusi, Nosema species, Pleistophora species, Thachipleistophora homin is, Vittaforma corneae, and Blastocystis hominis.

As used herein, an “antibiotic-resistant bacterium,” is a bacterium that is a member of a species of bacteria that has historically exhibited greater susceptibility to one or more particular antibiotic agents than the antibiotic-resistant member bacterium presently exhibits.

### EXPERIMENTAL EXAMPLES

### Example 1

An experiment was conducted to evaluate the antimicrobial activity of nitric oxide delivered in combination with oxygen, as compared with the antimicrobial activity of nitric oxide delivered without oxygen. Full-thickness porcine wounds were infected with bacteria, and then treated with nitric oxide in combination with oxygen or with nitric oxide without oxygen. Infected wounds were treated with either 10,000 ppm nitric oxide without oxygen for 30 minutes, 10,000 ppm nitric oxide in combination with 20% oxygen for 30 minutes, or 10,000 ppm nitric oxide in combination with 20% oxygen for 120 minutes. After treatment, biopsies of infected and treated wounds, as well as of infected but untreated wounds, were taken and a bacterial population count from each biopsy was determined.

A pathogen-free, commercially-raised, female, Yorkshire-cross pig weighing about 20 to 30 kg (Real Hog Farm, Marion, Tex.) was used. Before and during therapy, the s pig was housed in a raised stainless steel pen cage (6′×6′). The pig was fed antibiotic-free feed (Lab Diet Mini-Pig HF grower, PMI Nutrition International Inc., Brentwood, Mo.) and provided tap water ad libitum. Prior to wound creation, the pig received a comprehensive health inspection, The pig was examined and found to be healthy and fit for the study; no evidence of dermal or respiratory pathogens was detected. Also, 24 hours post wound infection, and just prior to the nitric oxide treatments, the general health and appearance of the pig was again monitored.

The day before wound creation, the pig was anesthetized with Telazol (Tiletamine/Zolazepam; 5 mg/kg, intramuscular; Fort Dodge Animal Health, Fort Dodge, Iowa). A small portion of the caudal dorsum was trimmed with a # 40 Oster clipper blade. A Fentanyl patch, Duragesic (2.5 ug/hr) (Fentanyl transdermal system, Watson Laboratories, Inc., Corona, Calif.), was secured to the shaved skin as post-surgical pain management. The pig was premedicated by intramuscular injection of Glycopyrrolate (0.003 mg/kg) (American Regent Inc., Shirley, N.Y.) follow'by Telazol (Tiletamine/Zolazepam; 5 mg/kg intramuscular; Fort Dodge Animal Health, Fort Dodge, Iowa) and followed by intubation and inhalation of 1 to 2 percent Isoflurane USP (Attane, Minrad Inc., Buffalo, N.Y.) mixed with oxygen. The dorsal and lateral thorax and abdomen of the pig was trimmed with a # 40 Oster clipper blade and washed with an antimicrobial-free soap.

On the day of wound creation (Day 0), the pig was transferred to a surgical suite and general anesthesia was continued. Blood was drawn immediately prior to wound creation and streaked on Tryptic Soy Broth (TSB), Pseudomonas Isolation Agar (PIA) and Mannitol Salts Agar (MSA) agar plates, to assess for the presence of bacteria in the blood.

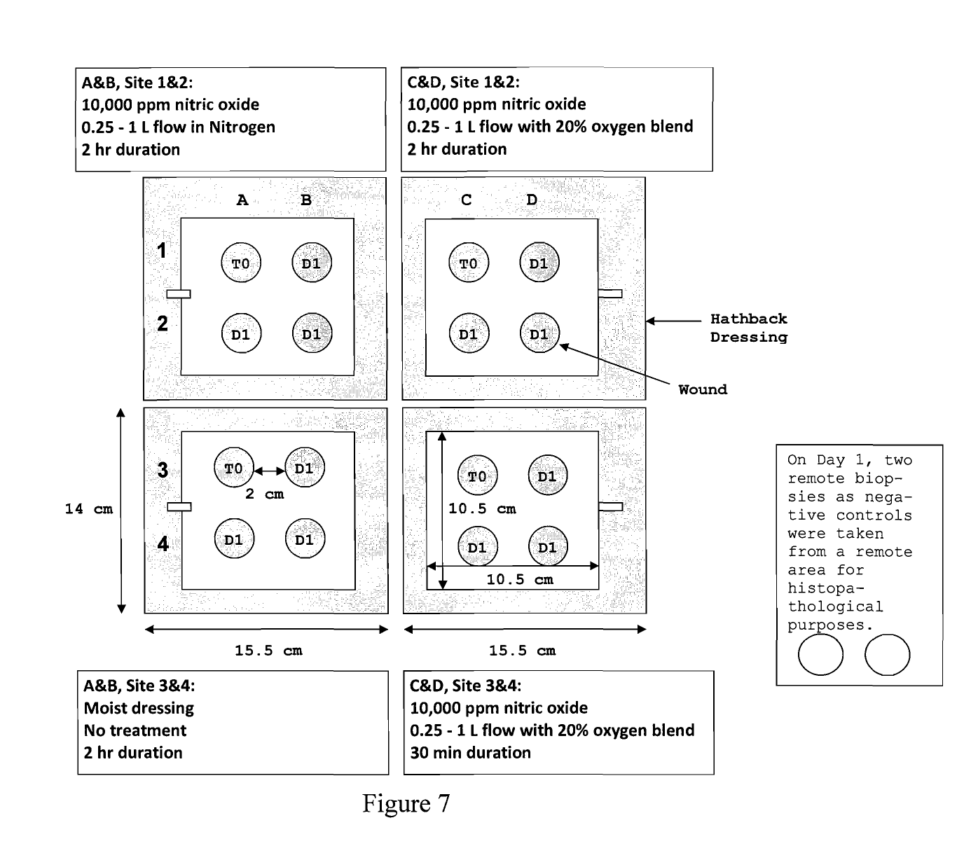

Sixteen full-thickness wounds, each about 20 mm in diameter, were created using custom-designed 2-cm trephine. Wounds were placed in groups of 4, with space left between the groups to allow for the proper later application of dressing materials. Additionally, each wound was placed so that at least about 2 cm remained between it and any other wound. See FIG. 7 for a picture depicting the arrangement of wounds.

Epinephrine solution (1:10,000 dilution) was applied using gauze sponges until hemostasis was achieved (approximately 10 minutes). The wounds were inoculated with coagulase-negative Staphylococci (CNS), which had been grown in standard growth media at 37° C. overnight prior to the day of wound creation. On the morning of wound creation, the CNS, grown to a density of 10̂10 CFU/mL, were washed with sterile saline and resuspended in saline to a final density of approximately 10̂6 CFU/mL. Sufficient inoculum was made to ensure that all the wounds could be saturated with the same preparation. CNS solution-saturated sponges were applied to the wounds. The sponges were left on the wounds and covered with an occlusive layer of Saran Wrap (S.C. Johnson & Sons, Brantford, ON) for 15 minutes. Then, the contaminated Saran Wrap and sponges were removed and discarded. All wounds were dressed using an absorbent dressing, Telfa™ (Covidien Mansfield, Mass.). Before applying the Telfa dressings, they were moistened with saline and squeezed to is remove excess saline. The Telfa dressings were secured in place with Transpore tape (3M, St. Paul, Minn.). All wounds were covered with a blue-absorbent pad as a secondary dressing. The absorbent layer of the blue pad was left in place for 24 hours. The pig was wrapped with a layer of elastic bandage over the blue pad to prevent movement of the dressings underneath. The pig was returned to its cage.

On Day 1, 24 hours after CNS inoculation, and just prior to commencing therapy, the pigs received another health assessment. Immediately prior to commencing therapy, a second blood sample was taken and streaked on TSB, PIA and MSA agar plates to assess for the presence of bacteria in the blood. The wounds were covered with modified Hathback Dressing and bathed with gas. See arrangement of treatment/control regime in FIG. 7. Columns A & B, sites 1 & 2: 120 minutes nitric oxide, balance nitrogen (i.e., no added oxygen). Columns C & D, sites 1 & 2: 120 minutes nitric oxide in combination with 20% oxygen. Columns C & D, sites 3 & 4: 30 minutes nitric oxide in combination with 20% oxygen. Columns A & B, sites 3 & 4, control, no treatment (moist dressing). Gas was delivered with a flow rate between 0.25 and 1 liter per minute.

After therapy, a third blood sample was taken, immediately upon conclusion of the 2 hour treatments with NO therapy and was streaked on TSB, PIA and MSA agar plates to assess for the presence of bacteria in the blood

On Day 1, twenty four hours after CNS inoculation and prior to therapy, 28 biopsies were taken; seven biopsies from each T0 wound (see FIG. 7). On Day 1 post therapy, 86 biopsies were taken, seven biopsies from each Dl wound and two biopsies from a remote area. Two biopsies were taken from inside and one from outside of each wound for microbiological sampling, and were made using a 4-mm biopsy punch. One inside biopsy was taken in the approximate center of the wound. A second inside biopsy was taken at a position of “12 o'clock” immediately inside (but not including) the wound edge. One biopsy from inside the wound and one biopsy from outside the wound was rinsed in 10% NBF to remove blood and placed in fresh for histopathology examination. Two biopsies were taken from remote areas as negative controls for histopathological purposes.

The biopsy tissues were placed into a pre-weighed vessel containing is phosphate buffered saline and the weight of tissue was determined. The biopsy samples were individually homogenized and serially diluted. After processing, the pre-therapy microbiology samples were plated. The serial dilutions were Drop-Plated and incubated to determine the bacterial counts. The samples were plated on TSB to determine the total number of bacteria present in the biopsy specimen. The samples were plated on MSA to determine the number of Staphylococci present in the biopsy specimen. Bacterial counts were expressed as log10 (CFU/g).

After processing, the post NO therapy microbiology samples were plated at two time points: immediately after processing and two hours after processing. At t=0 hours, the serial dilutions were Drop-Plated and incubated to determine the bacterial counts. The samples were plated on TSB to determine the total number of bacteria present in the biopsy specimen. The samples were plated on MSA to determine the number of staphylococci present in the biopsy specimen. Bacterial counts were expressed as log 10 (CFU/g). At t=2 hours, the serial dilutions were Drop-Plated following a 2-hour incubation in saline, and then incubated to determine the bacterial counts, The samples were plated on TSB to determine the total number of bacteria present in the biopsy specimen. The samples were plated on MSA to determine the number of staphylococci present in the biopsy specimen. Bacterial counts were expressed as log 10 (CFU/g).

All health inspections showed the pig was healthy and acceptable for the study. All blood samples were taken during the study and streaked on TSA, MSA, and PIA culture plates verified that the blood was free of bacterial contamination.

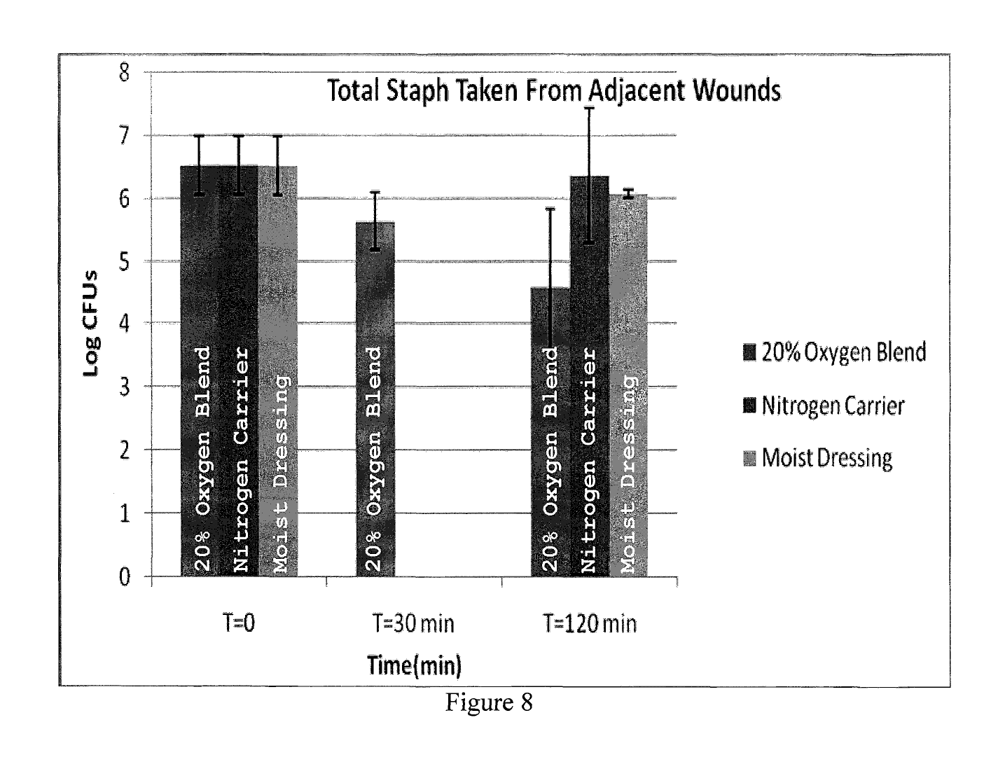

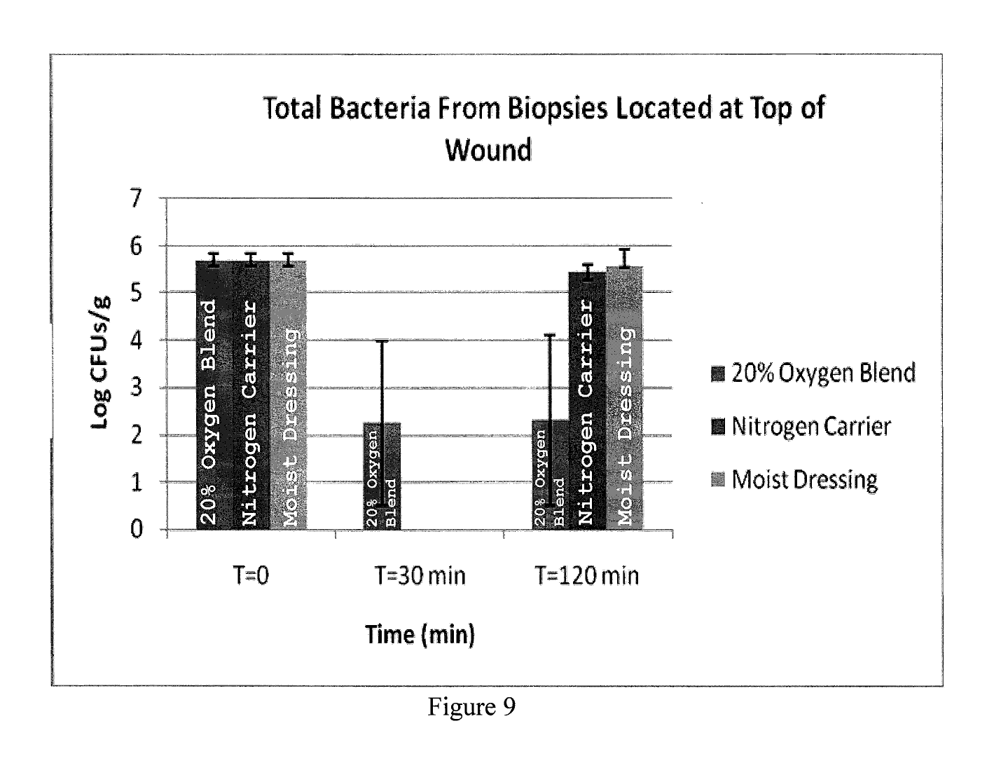

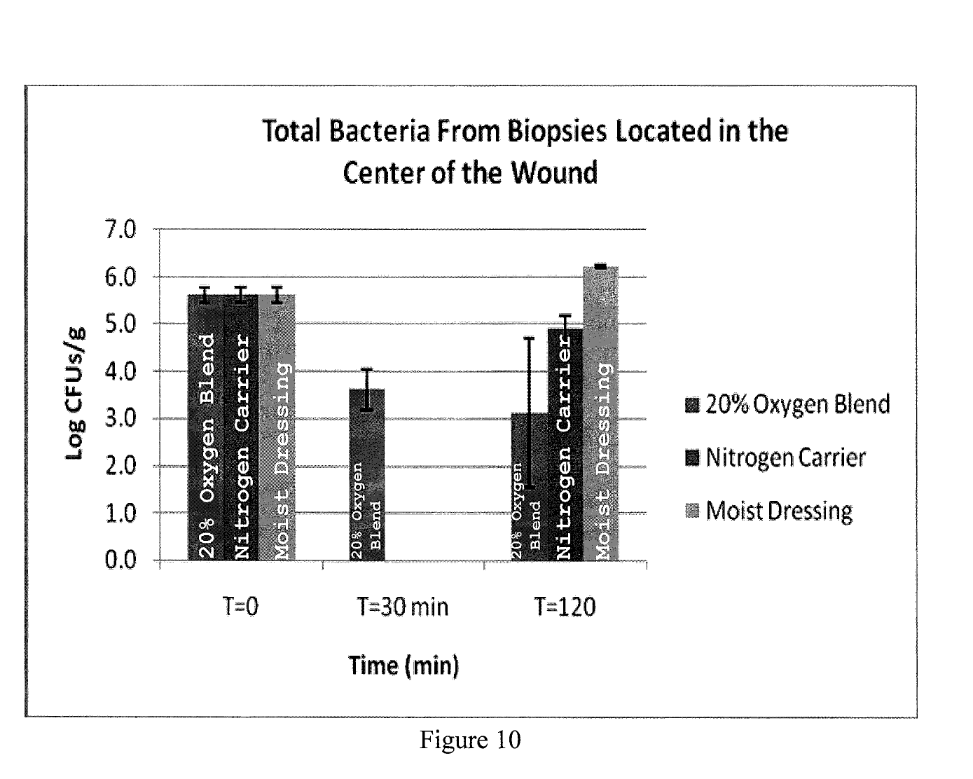

The nitric oxide in combination with 20% oxygen revealed a significant reduction (p<0.05) in Staphylococcus from biopsies taken adjacent to the wound in relation to length of treatment (FIG. 8). There was no significant difference in total amount of bacteria (those plated on TSA) in the adjacent biopsies related to length of time of treatment (data not shown). There was also a significant decrease (p<0.05) in amount of total bacteria from biopsies taken from the top of wounds given the nitric oxide in combination with 20% oxygen therapy (FIG. 9). In biopsies taken from the center of the wounds, there was also a significant decrease (p<0.05) in the amount of total bacteria when the wounds were treated with nitric oxide in combination with 20% oxygen (FIG. 10). There was a significant decrease (p<0.05) in the amount of total bacteria in biopsies from the center of wounds treated with nitric oxide in the nitrogen carder (FIG. 10), but on average there was a smaller decrease as compared with the decrease of nitric oxide in combination with oxygen. All the moist control biopsies showed a significant increase in amount of bacteria (p<0.05). The decrease in bacteria did not continue after biopsy samples were processed for plating and left to sit for two hours before plating (data not shown).

### Example 2

Experiments were conducted to evaluate the antimicrobial activity of nitric oxide delivered in combination with varying concentrations of oxygen. Full-thickness porcine wounds were infected with bacteria, and then treated with nitric oxide in combination with varied concentrations of oxygen. Two pigs were used. For pigs 1 and 2, infected wounds were treated with either 10,000 ppm nitric oxide in combination with 10% oxygen for 30 minutes, 10,000 ppm nitric oxide in combination with 15% oxygen for 30 minutes, or 10,000 ppm nitric oxide in combination with 20% oxygen for 30 minutes. After treatment, biopsies of infected and treated wounds, as well as of infected but untreated wounds, were taken and a bacterial population count from each biopsy was determined in duplicate.

Pathogen-free, commercially-raised, female, Yorkshire-cross pigs weighing about 20 to 30 kg (Real Hog Farm, Marion, Tex.) were used. Before and during therapy, the pigs were housed in a raised stainless steel pen cage (6′×6′). The pigs were fed antibiotic-free feed (Lab Diet Mini-Pig HF grower, PMI Nutrition International Inc., Brentwood, Mo.) and provided tap water ad libitum. Prior to wound creation, the pigs received a comprehensive health inspection. The pigs were examined and found to be healthy and fit for the study; no evidence of dermal or respiratory pathogens was detected. Also, 24 hours post wound infection, and just prior to the nitric oxide treatments, the general health and appearance of the pigs was again monitored.

The day before wound creation, the pigs were anesthetized with Telazol (Tiletamine/Zolazepam; 5 mg/kg, intramuscular; Fort Dodge Animal Health, Fort Dodge, Iowa). A small portion of the caudal dorsum was trimmed with a # 40 Oster clipper blade. A is Fentanyl patch, Duragesic (2.5 ug/hr) (Fentanyl transdermal system, Watson Laboratories, Inc., Corona, Calif.), was secured to the shaved skin as post-surgical pain management. The pigs were premedicated by intramuscular injection of Glycopyrrolate (0.003 mg/kg) (American Regent Inc., Shirley, N.Y.) follow by Telazol (Tiletamine/Zolazepam; 5 mg/kg intramuscular; Fort Dodge Animal Health, Fort Dodge, Iowa) and followed by intubation and inhalation of 1 to 2 percent Isoflurane USP (Attane, Minrad Inc., Buffalo, N.Y.) mixed with oxygen. The dorsal and lateral thorax and abdomen of the pigs was trimmed with a # 40 Oster clipper blade and washed with an antimicrobial-free soap.

On the day of wound creation (Day 0), the pigs were transferred to a surgical suite and general anesthesia was continued. Blood was drawn immediately prior to wound creation and streaked on TSB, PIA and MSA agar plates to assess for the presence of bacteria in the blood.

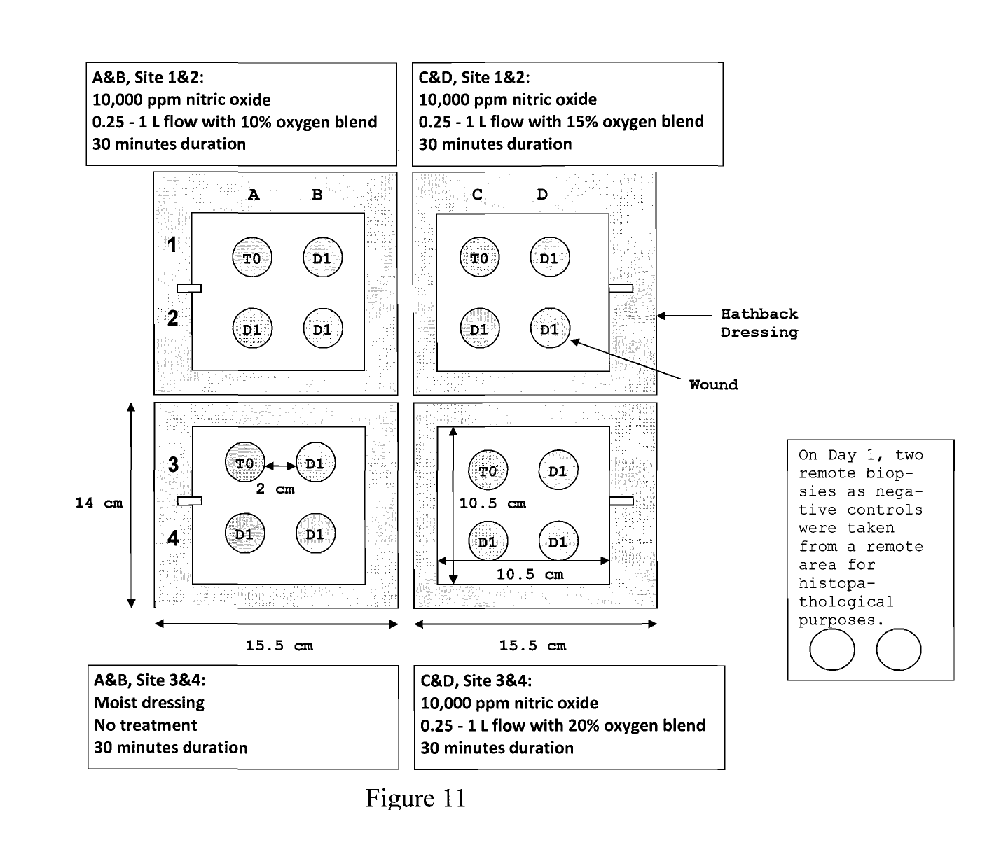

On each of the pigs, sixteen full-thickness wounds, each about 20 mm in diameter, were created using custom-designed 2-cm trephine. Wounds were placed in groups of 4, with space left between the groups to allow for the proper later application of dressing materials. Additionally, each wound was placed so that at least about 2 cm remained between it and any other wound. See FIG. 11 for a picture depicting the arrangement of wounds.

Epinephrine solution (1:10,000 dilution) was applied using gauze sponges until hemostasis was achieved (approximately 10 minutes). The wounds were inoculated with coagulase-negative Staphylococci (CNS), which had been grown in standard growth media at 37° C. overnight prior to the day of wound creation. On the morning of wound creation, the CNS, grown to a density of 10\textcircled{}10 CFU/mL, were washed with sterile saline and resuspended in saline to a final density of approximately 10̂6 CFU/mL. Sufficient inoculum was made to ensure that all the wounds could be saturated with the same preparation. CNS solution-saturated sponges were applied to the wounds. The sponges were left on the wounds and covered with an occlusive layer of Saran Wrap (S.C. Johnson & Sons, Brantford, ON) for 15 minutes. Then, the contaminated Saran Wrap and sponges were removed and discarded. All wounds were dressed using an absorbent dressing, Telfa™ (Covidien Mansfield, Mass.). Before applying the Telfa dressings, they were moistened with saline and squeezed to is remove excess saline. The Telfa dressings were secured in place with Transpore tape (3M, St. Paul, Minn.). All wounds were covered with a blue-absorbent pad as a secondary dressing. The absorbent layer of the blue pad was left in place for 24 hours. The pigs were wrapped with a layer of elastic bandage over the blue pad to prevent movement of the dressings underneath. The pigs were returned to their cages.

On Day 1, 24 hours after CNS inoculation, and just prior to commencing therapy, the pigs received another health assessment. Immediately prior to commencing therapy, a second blood sample was taken and streaked on TSB, PIA and MSA agar plates to assess for the presence of bacteria in the blood. The wounds were covered with modified Hathback Dressing and bathed with gas. See arrangement of treatment/control regime in FIG. 11 Columns A & B, sites 1 & 2: 30 minutes nitric oxide in combination with 10% oxygen. Columns C & D, sites 1 & 2: 30 minutes nitric oxide in combination with 15% oxygen. Columns C & D, sites 3 & 4: 30 minutes nitric oxide in combination with 20% oxygen. Columns A & B, sites 3 & 4, control, no treatment (moist dressing). Gas was delivered with a flow rate between 0.25 and 1 liter per minute.

Immediately after therapy, a third blood sample was taken and streaked on TSB, PIA and MSA agar plates to assess for the presence of bacteria in the blood

On Day 1, twenty four hours after CNS inoculation and prior to therapy, from each of the pigs, 28 biopsies were taken; seven biopsies from each T0 wound (see FIG. 11). On Day 1 post therapy, 86 biopsies were taken, seven biopsies from each D1 wound and two biopsies from a remote area. Two biopsies were taken from inside and one from outside of each wound for microbiological sampling, and were made using a 4-mm biopsy punch. One inside biopsy was taken in the approximate center of the wound. A second inside biopsy was taken at a position of “12 o'clock” immediately inside (but not including) the wound edge. One biopsy from inside the wound and one biopsy from outside the wound was rinsed in 10% NBF to remove blood and placed in fresh for histopathology examination. Two biopsies were taken from remote areas as negative controls for histopathological purposes.

The biopsy tissues were placed into a pre-weighed vessel containing phosphate buffered saline and the weight of tissue was determined. The biopsy samples were individually homogenized and serially diluted. After processing, each serial dilution was Drop-Plated and incubated on two separate Tryptic Soy Agar (TSA) plates to determine the bacterial count. Bacterial counts were expressed as log 10 (CFU/g).

All health inspections showed the pigs were healthy and acceptable for the study. All blood samples were taken during the study and streaked on TSA, MSA, and PIA culture plates verified that the blood was free of bacterial contamination.

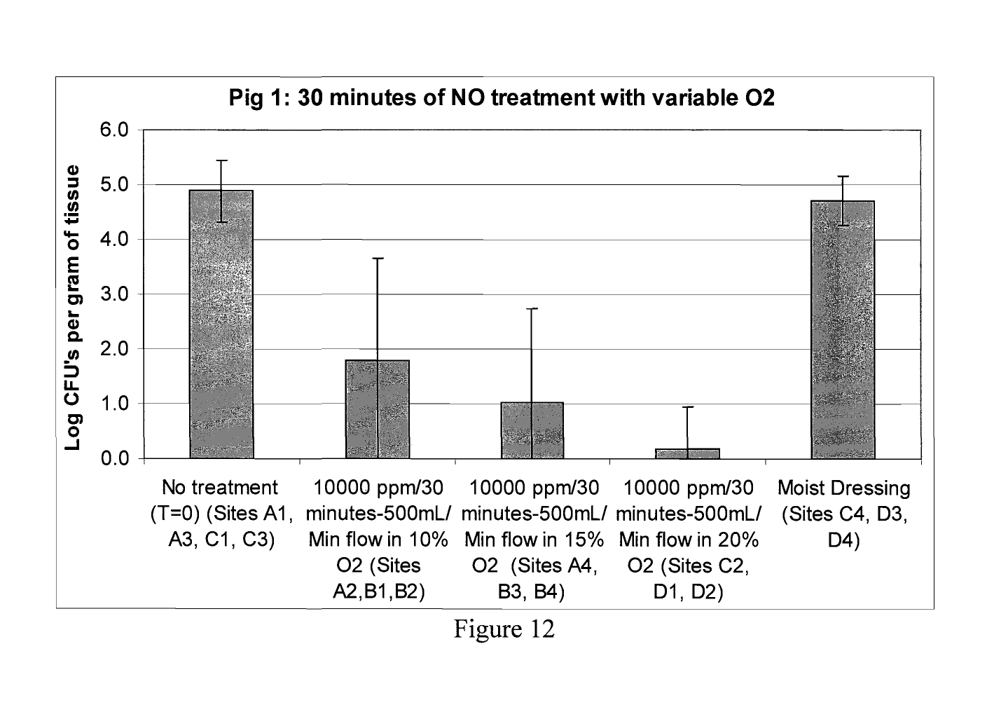

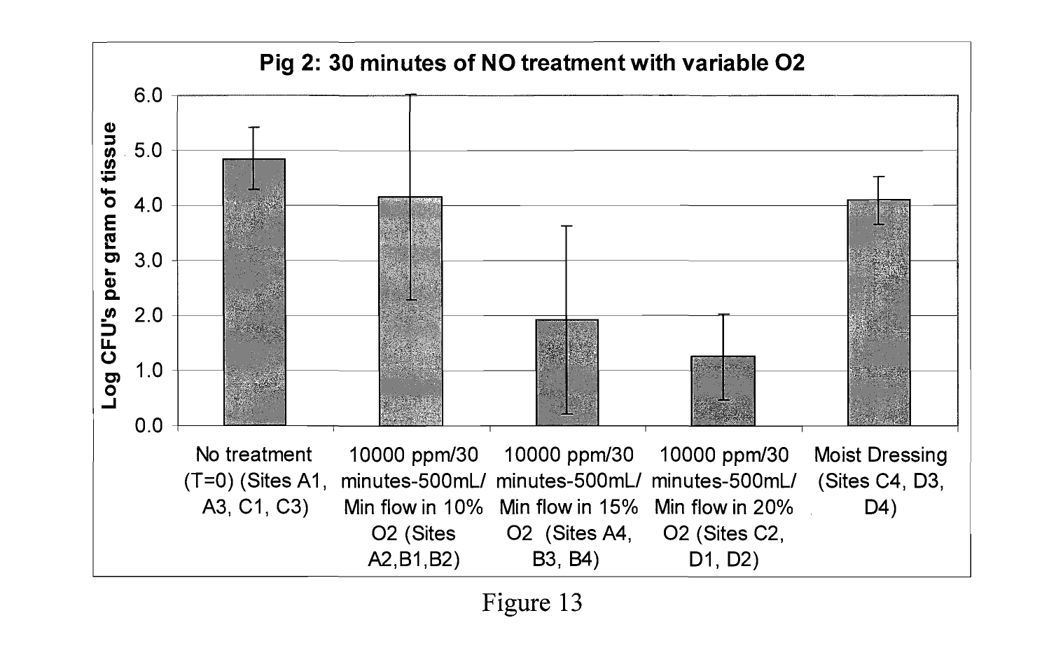

An average of bacterial counts determined from all biopsies taken from all wounds in each treatment group are depicted in FIGS. 12-13. For pigs 1 and 2, treatment with 10,000 ppm nitric oxide in combination with oxygen for 30 minutes reduced bacterial counts in biopsy samples, as compared with bacterial counts of wound biopsy samples from untreated or moist-dressing treated wounds (see FIGS. 12 and 13). Moreover, treatment with 10,000 ppm nitric oxide in combination with 20% oxygen reduced bacterial counts greater than treatment in combination with 15% oxygen, which reduced bacterial counts greater than treatment in combination with 10% oxygen (see FIGS. 12 and 13).

### Example 3

To generate a gas mixture containing 18,000 ppm nitric oxide and 20% oxygen, nitric oxide gas, from a source of nitric oxide gas having a concentration of 23,000 ppm, is mixed with oxygen gas, from a source of oxygen gas having a concentration of 100%, and with a dilutant gas, from a source of dilutant gas (e.g., N₂) having a concentration of 100%, in the following ratio (NO:O₂:dilutant): 0.7826 0.2000 0.0174.

### Example 4

To generate a gas mixture containing 15,000 ppm nitric oxide and 20% oxygen, nitric oxide gas, from a source of nitric oxide gas having a concentration of 23,000 ppm, is mixed with oxygen gas, from a source of oxygen gas having a concentration of 100%, and with a dilutant gas, from a source of dilutant gas (e.g., N₂) having a concentration of 100%, in the following ratio (NO:O₂:dilutant): 0.6522:0.2000:0.1478.

### Example 5

To generate a gas mixture containing 10,000 ppm nitric oxide and 20% oxygen, nitric oxide gas, from a source of nitric oxide gas having a concentration of 23,000 ppm, is mixed with oxygen gas, from a source of oxygen gas having a concentration of 100%, and with a dilutant gas, from a source of dilutant gas (e.g., N₂) having a concentration of 100%, in the following ratio (NO:O₂:dilutant): 0.4347:0.2000:0.3652.

### Example 6

To generate a gas mixture containing 5,000 ppm nitric oxide and 20% oxygen, nitric oxide gas, from a source of nitric oxide gas having a concentration of 23,000 ppm, is mixed with oxygen gas, from a source of oxygen gas having a concentration of 100%, and with a dilutant gas, from a source of dilutant gas (e.g., N₂) having a concentration of 100%, in the following ratio (NO:O₂:dilutant): 0.2174:0.2000:0.5826.

### Example 7

To generate a gas mixture containing 30,000 ppm nitric oxide and 20% oxygen, nitric oxide gas, from a source of nitric oxide gas having a concentration of 40,000 ppm, is mixed with oxygen gas, from a source of oxygen gas having a concentration of 100%, and with a dilutant gas, from a source of dilutant gas (e.g., N₂) having a concentration of 100%, in the following ratio (NO:O₂:dilutant): 0.7500:0.2000:0.0500.

### Example 8

To generate a gas mixture containing 20,000 ppm nitric oxide and 20% oxygen, nitric oxide gas, from a source of nitric oxide gas having a concentration of 40,000 ppm, is mixed with oxygen gas, from a source of oxygen gas having a concentration of 100%, and with a dilutant gas, from a source of dilutant gas (e.g., N₂) having a concentration of 100%, in the following ratio (NO:O₂:dilutant): 0.5000:0.2000:0.0300.

### Example 9

To generate a gas mixture containing 10,000 ppm nitric oxide and 20% oxygen, nitric oxide gas, from a source of nitric oxide gas having a concentration of 40,000 ppm, is mixed with oxygen gas, from a source of oxygen gas having a concentration of 100%, and with a dilutant gas, from a source of dilutant gas (e.g., N₂) having a concentration of 100%, in the following ratio (NO:O₂:dilutant): 0.2500:0.2000:0.5500.

### Example 10

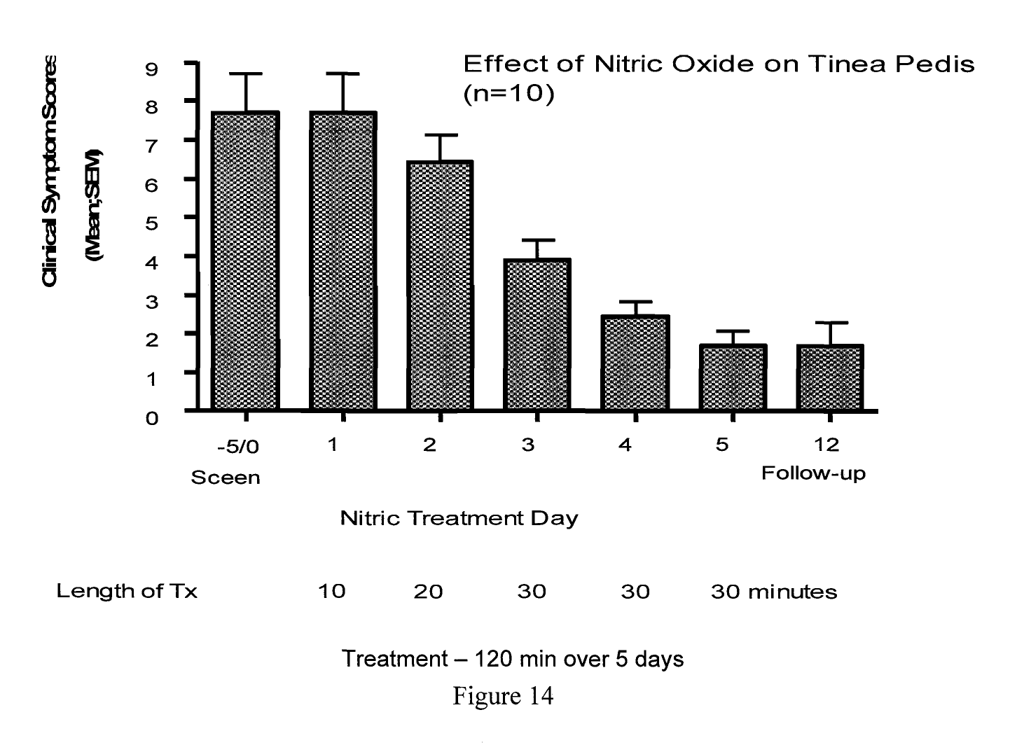

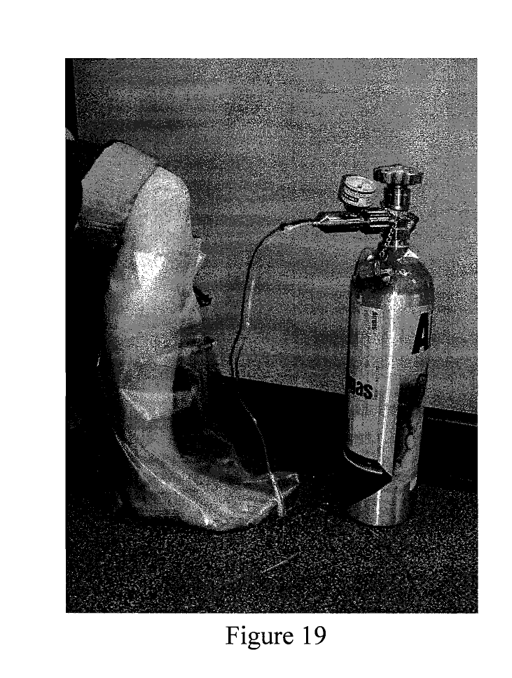

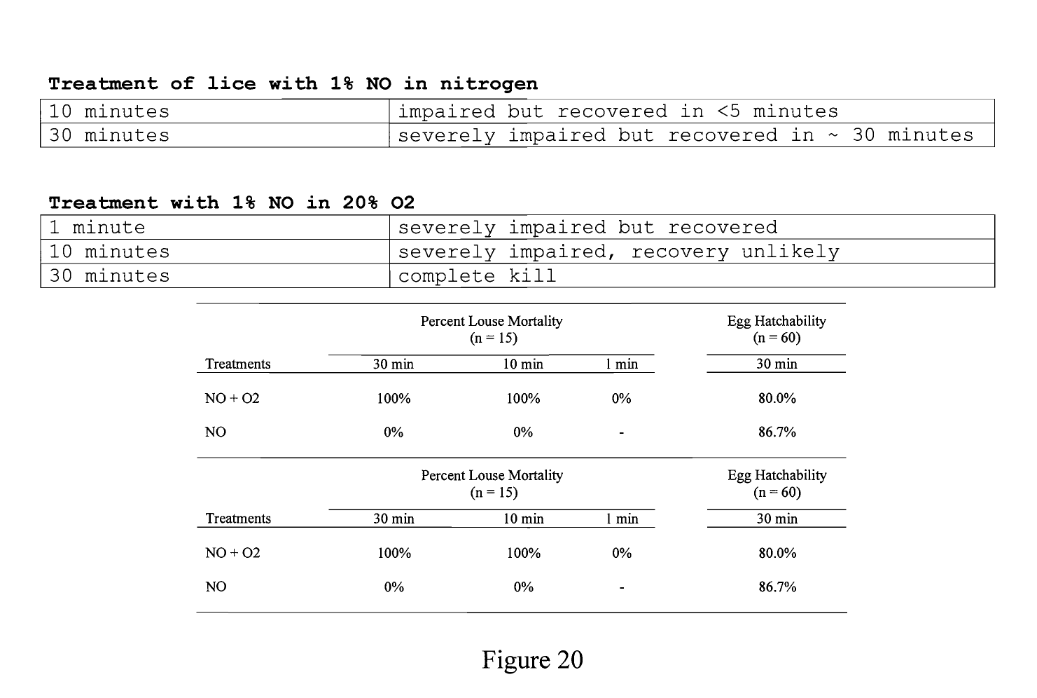

To evaluate the effectiveness of gaseous nitric oxide treatment against mild tinea pedis, a single-site clinical trial treating 10 human patients with mild tinea pedis was conducted. Subjects who had a pre-existing chronic systemic dermal disease, other than tinea in, or immediately around the area under evaluation were excluded from this study. The foot of each patient was fitted with a flexible, plastic boot-like enclosure that was sealed at the mid-calf (see, for example, FIG. 19) and the foot was bathed with 1% gaseous nitric oxide in nitrogen at a fixed flow of 1 liter per minute for a total of 2 hours divided over 5 treatment days. Gas entered the boot-like enclosure near the toe region with passive diffusion from the enclosure into a well-ventilated examination room. On day 1, patients were treated for 10 minutes. On day 2, patients were treated for 20 minutes. On each of days 3, 4 and 5, patients were treated for 30 minutes. On days 0, 1, 2, 3, 4, 5 and 12, each patient was assessed by a podiatrist blinded to the patient's treatment group and given a clinical symptom severity score ranging from 0 to 64. The clinical symptom severity score is determined by scoring each of 8 categories (i.e., fissures/cracks, yellow crusting (ruptured blisters), maceration (white moist skin), pruritus (itching), scaling, erythema, vesicles/blisters and burning) on an 8-point scale (i.e., 0=absent, 1-2 mild, 3-5 moderate and 6-8 severe). The mean clinical symptom severity score among all 10 patients on each of days 0, 1, 2, 3, 4, 5 and 12, is depicted in FIG. 14. Average clinical symptom scores began to decrease on the second day of treatment and continued to decrease until the fifth day of treatment. On day 12, seven days after the last treatment, the average clinical symptom severity score remained as low as it was on day 5.

### Example 11

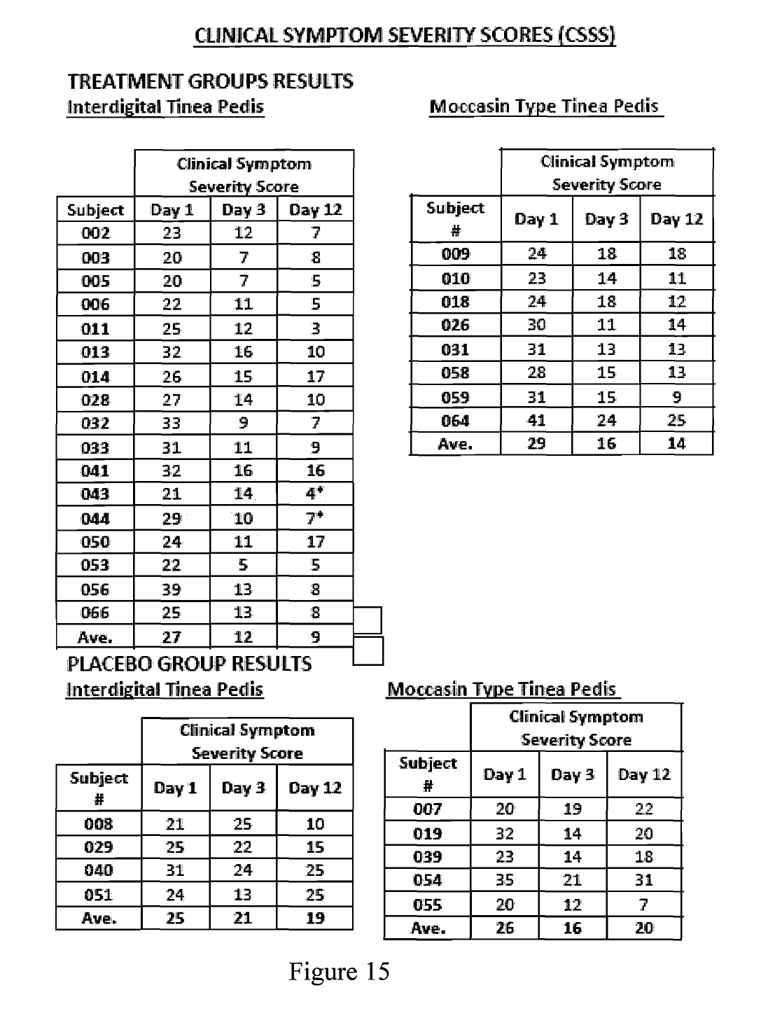

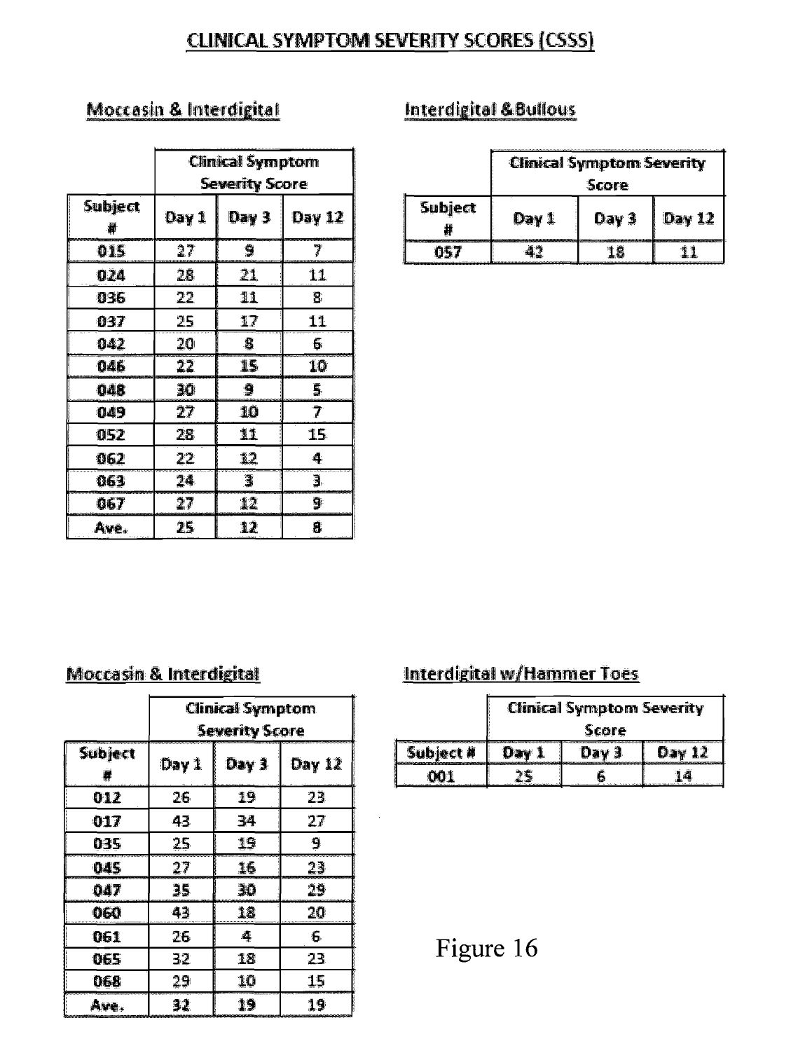

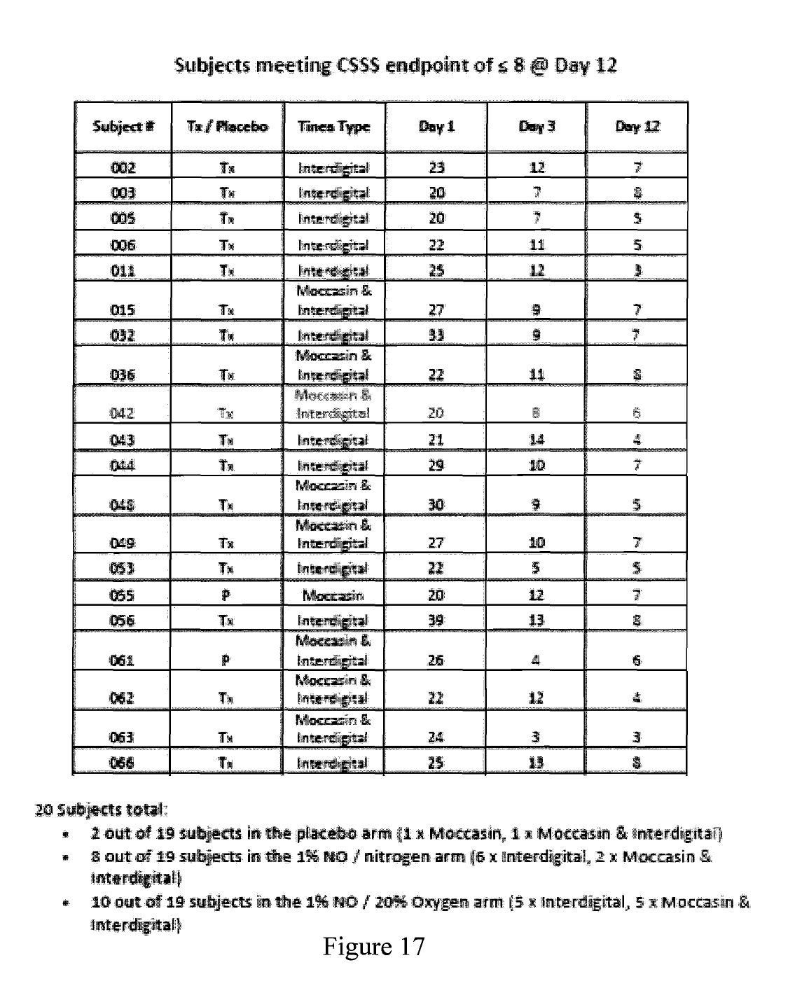

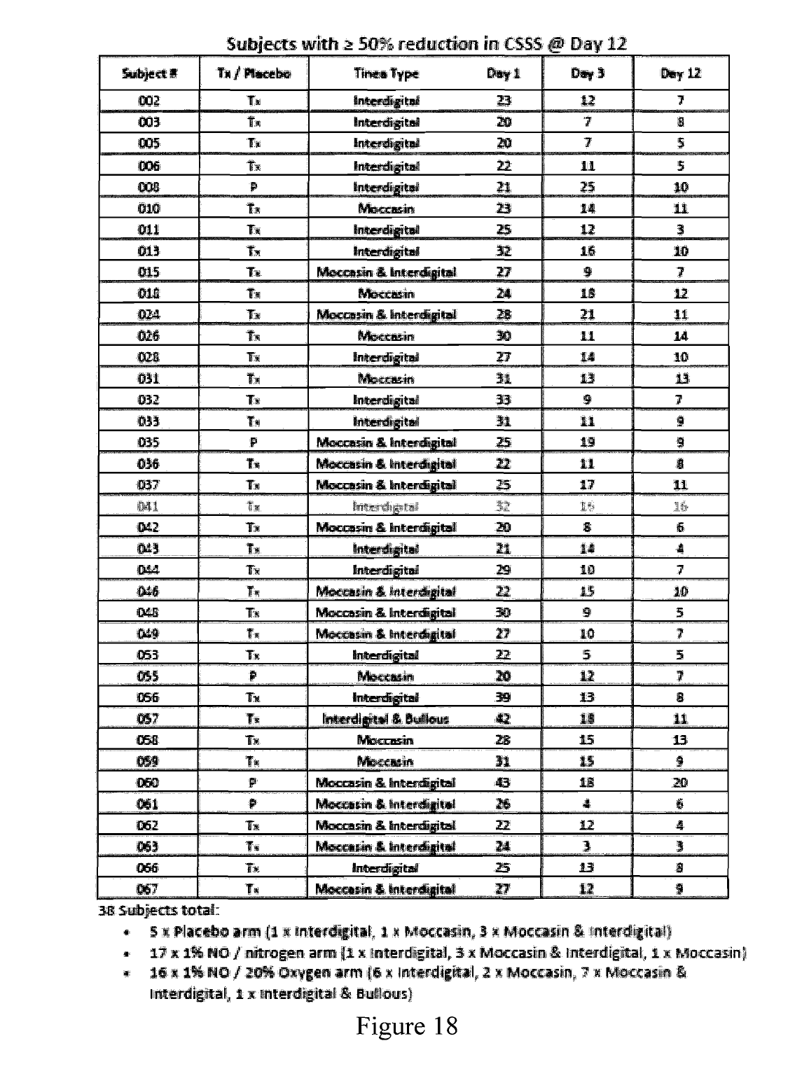

To evaluate the effectiveness of gaseous nitric oxide treatment against moderate-severe interdigital and/or moccasin-type tinea pedis, a single-site, placebo-controlled clinical trial treating≧50 evaluable human patients with moderate-severe interdigital tinea pedis was conducted. Subjects who had a pre-existing chronic systemic dermal disease, other than tinea in, or immediately around the area under evaluation were excluded from this study. The foot of each patient was fitted with a flexible, plastic boot-like enclosure that was sealed at the mid-calf (see, for example, FIG. 19) and the foot was bathed with 1% gaseous nitric oxide in nitrogen, or 1% gaseous nitric oxide and 20% oxygen in nitrogen, or placebo (i.e., nitrogen) at a fixed flow of 1 liter per minute for 40 minutes each day for each of three consecutive days, Gas entered the boot-like enclosure near the toe region with passive diffusion from the enclosure into a well-ventilated examination room. Each foot of each patient was fitted with an air-tight, ankle-high covering and the feet were On days 1, 3 and 12, each patient was assessed by a podiatrist blinded to the patient's treatment group and given a clinical symptom severity score ranging from 0 to 64. The clinical symptom severity score was determined by scoring each of 8 categories (i.e., fissures/cracks, yellow crusting (ruptured blisters), maceration (white moist skin), pruritus (itching), scaling, erythema, vesicles/blisters and burning) on an 8-point scale (i.e., 0=absent, 1-2 mild, 3-5 moderate and 6-8 severe), The clinical symptom scores of patients treated with 1% nitric oxide or with placebo are shown in FIGS. 15, 16, 17, and 18. For all patients treated with 1% nitric oxide, the clinical symptom severity score was lower on day 3 than on day 1. Most patients treated with 1% nitric oxide had a lower clinical symptom severity score on day 12 than on day 3. Eleven of 25 patients treated with 1% nitric oxide reached the target clinical endpoint score of ≦8 by day 12. Of the 13 patients with interdigital tinea, 8 reached the primary clinical endpoint score of ≦8, while 3 of the remaining 5 patient scores dropped more than 60% (data not shown). No placebos achieved the endpoint.

### Example 12

The percutaneous absorption of nitric oxide, in vitro, is evaluated using a Franz human skin finite dose model. The in vitro Franz human skin finite dose model has proven to be a valuable tool for the study of percutaneous absorption and the determination of the pharmacokinetics of topically applied drugs. The model uses human ex vivo cadaver or surgical skin mounted in a specially designed diffusion chamber allowing the skin to be maintained at a temperature and humidity that match typical in vivo conditions. A finite dose of a formulation is applied to the outer surface of the skin and drug absorption is measured by monitoring its rate of appearance in the reservoir solution bathing the inner surface of the skin. In this study, the applied dose is a controlled continuous exposure to a gas. Data defining total absorption, rate of absorption, as well as skin content can be accurately determined in this model. The method has historic precedent for accurately predicting in vivo percutaneous absorption kinetics (Franz, 1978, Skin: Drug Application and Evaluation of Environmental Hazards, Current Problems in Dermatology, vol. 7, G. Simon, Z. Paster, M. Klingberg, M. Kaye (Eds), Basel, Switzerland, S. Karger, 1978, pp 58-68).

A single center, open label study of two test formulations (i.e., 1% nitric oxide in nitrogen, and 1% nitric oxide, in combination with 20% oxygen, in nitrogen) is performed. The formulations are tested in triplicate on at least one skin donors using the in vitro Franz finite dose skin model (see Franz, 1975, Percutaneous absorption: on the relevance of in vitro data. J Invest Derm 64:190-195). The rate and extent of penetration of nitric oxide, at 1 atmosphere of pressure and at greater than 1 atmosphere of pressure (e.g,, 1.2×, 1.5×, 1.8×, 2.0×, 2.2×, etc.) for continuous exposure durations of 30 and 60 minutes is determined for the amount penetrating through the skin and into the different layers of the skin using a Franz Diffusion Cell.

Human cadaver trunk skin is used in this study. It is dermatomed, cryopreserved, sealed in a water-impermeable plastic bag, and stored at −70° C. until the day of the experiment. Prior to use it is thawed in 37° C. water, then rinsed in tap water to remove any adherent blood or other material from the surface.

Skin from a single donor is cut into multiple smaller sections large enough to fit on, for example, a 1.0 cm² Franz diffusion cell. To assure the integrity of each skin section, its permeability to tritiated water is determined before application of the test products (Franz and Lehman, 1990, Abst. J Invest Dermatol 1990, 94:525). Following a brief (0.5-1 hour) equilibrium period, ³H₂0 (NEN, Boston, Mass., sp. Act.—0.5 pCi/mL) is layered across the top of the skin by dropper so that the entire exposed surface is covered (approximately 200-500 gL). After 5 minutes, the ³H₂0 aqueous layer is removed. At 30 minutes, the reservoir solution is collected and analyzed for radioactive content by liquid scintillation counting. Skin specimens in which absorption of ³H₂0 is less than 1 56 pi, equ/cm² are considered acceptable.

The dermal chamber is filled to capacity with a reservoir solution of phosphate-buffered isotonic saline (PBS), pH 7.4 0.1, and the epidermal chamber is left open to ambient laboratory environment. The cells are placed in a diffusion apparatus in which the dermal reservoir solution is stirred magnetically at 600 RPM and its temperature maintained to achieve a skin surface temperature of 32.0±1.0° C. The formulations are tested and the rate and extent of penetration of nitric oxide, at I atmosphere of pressure and at greater than 1 atmosphere of pressure (e.g., 1.2×, 1.5×, 1.8×, 2.0×, 2.2×, etc.) for continuous exposure durations of 30 and 60 minutes is determined for the amount penetrating through the skin and into the different layers of the skin using a Franz Diffusion Cell. Pharmacokinetic sampling of the reservoir solution occurs prior to dosing (0 hour) and, for example, at 10, 15, 30, 60 and 120 minutes during and following gas exposure. At the end of the dose duration, and following the collection of the last receptor solution, the surface of the skin sections are washed and the stratum corneum, epidermis, and dermis are isolated and each are retained for analysis. Nitric oxide concentrations are measured using an analytical method.

### Example 13

The ability of nitric oxide or nitric oxide+oxygen to kill human head louse females and eggs was evaluated using gas chamber bioassay.

The methods and materials of the example are now described.

Human head lice from the permethrin(kdr)-resistant SF-HL strain were used in a nitric oxide gas bioassay to determine louse mortality at different exposure intervals. Adult female lice and aged eggs (6-8 day old) were used in all bioassays. The lice or eggs were placed in the bottom of a sterile disposable Petri dishes (60×20 mm) and left uncovered. The dish with lice or eggs was placed inside a polyethylene bag and the bag was taped shut with HY-TAPE® (HY-TAPE Intl., Patterson, N.Y.) to seal the chamber and to minimize the volume of gas inside the chamber. The treatments used were either nitric oxide, in combination with oxygen mixed in equal parts at a final concentration of 10,000 ppm nitric oxide and 20% oxygen, or a treatment with nitric oxide alone at 10,000 ppm in nitrogen both with a flow rate of 1 liter/minute. The gas tank was connected to the chamber with PVC tubing and plastic connectors. The nitric oxide, in combination with oxygen, was mixed in the tubing before the chamber inlet. The chamber outlet was also connected with PVC tubing and fed to a NO scrubber to trap the NO gas leaving the chamber. Additionally, tufts of human hair (blond and brown) were placed inside the chamber for a 30 minute exposure interval to nitric oxide+oxygen to assess discoloration of the hair for cosmetic purposes.

The results of the example are now described.

Quantitative and qualitative results of an example experiment are depicted in the FIG. 19. Nitric oxide, in combination with oxygen, was able to kill 100% of lice treated using a 10 minute or 30 minute exposure interval. At a 1 minute exposure interval, nitric oxide, in combination with oxygen, did not kill any of the lice treated. Also, nitric oxide, in combination with oxygen, was not ovicidal to aged eggs (6-8 days old) using a 30 minute exposure interval. Egg hatchability was not different from untreated control eggs.

Nitric oxide alone did not kill any of the lice treated using a 10 minute or 30 minute exposure interval. Also, nitric oxide alone was not ovicidal to aged eggs (6-8 days old) using a 30 minute exposure interval. Egg hatchability was not different from untreated control eggs.

The disclosures of each and every patent, patent application, and publication cited herein are hereby incorporated herein by reference in their entirety.

While this invention has been disclosed with reference to specific embodiments, it is apparent that other embodiments and variations of this invention may be is devised by others skilled in the art without departing from the true spirit and scope of the invention. The appended claims are intended to be construed to include all such embodiments and equivalent variations.

What is claimed:

1. A method of delivering a gas mixture containing an effective amount of nitric oxide, in combination with oxygen, to an infected site of a patient to reduce pathogen levels comprising the steps of:
providing a flow-controlled source of a gas containing nitric oxide and a flow-controlled source of a gas containing oxygen;
mixing the gas containing nitric oxide with the gas containing oxygen, wherein the gas mixture contains at least about 10,000 ppm nitric oxide and at least about 20% oxygen;
providing a bathing unit around the infected site of the patient, the bathing unit forming a seal with the infected site of the patient;
transporting the gas mixture to the bathing unit so as to bathe the infected site of the patient with the gas mixture.

2. The method of claim 1, wherein the pathogen is at least one selected from the group consisting of a bacterium, a virus, a fungus, a parasite, an arthropod and a protozoan.

3. The method of claim 1, wherein the pathogen is an antibiotic-resistant bacteria.

4. The method of claim 3, wherein the antibiotic-resistant bacteria is at least one selected from the group consisting of Staphylococcus aureus, Pseudomonas aeruginosa, Streptococcus pneumoniae, Escherichia coli, Salmonella, Klebsiella, Enterococci, and combinations thereof.

5. The method of claim 1, further comprising the step of evacuating at least a portion of the nitric oxide gas is evacuated from the bathing unit.

6. The method according to claim 1, further comprising the step of removing at least a portion of the nitric oxide contained within the gas mixture that is evacuated from the bathing unit.

7. The method according to claim 1, further comprising the step of controlling the flow rate of gas mixture into and out of the bathing unit.

8. The method according to claim 1, further comprising the step of agitating the gas mixture within the bathing unit.

9. The method according to claim 1, further comprising the step of directing the flow of gas mixture onto the infected site of the patient.

10. The method of claim 1, wherein the infected site is a wound.

11. The method of claim 10, wherein the wound is at least one selected from the group consisting of a surgical wound, a trauma wound and a burn.

12. The method of claim 1, wherein the infected site is an abscess.

13. The method of claim 1, wherein the infected site is a lesion.

14. The method of claim 1, wherein the gas mixture is delivered to the infected site at a pressure greater than 1 atmosphere.

15. A method to promote healing of an area of the body of a patient with a wound, the method comprising the steps of: providing a flow-controlled source of a gas containing nitric oxide and a flow-controlled source of a gas containing oxygen; and mixing the gas containing nitric oxide with the gas containing oxygen, wherein the gas mixture contains at least about 10,000 ppm nitric oxide and at least about 20% oxygen; and delivering gas mixture to the area of the body so as to bathe the wound with the gas mixture; and wherein the wound is infected by at least one type of pathogen.

16. The method of claim 15, wherein the pathogen is at least one selected from the group consisting of a bacterium, a virus, a fungus, a parasite, an arthropod and a protozoan.

17. The method of claim 15, wherein the pathogen is an antibiotic-resistant bacteria.

18. The method of claim 15, wherein the antibiotic-resistant bacteria is at least one selected from the group consisting of Staphylococcus aureus, Pseudomonas aeruginosa, Streptococcus pneumoniae, Escherichia coli, Salmonella, Klebsiella, Enterococci, and combinations thereof.

19. The method of claim 15, wherein the gas mixture is delivered to the area using a bathing unit surrounding the area.

20. The method of claim 15, wherein the wound is at least one selected from the group consisting of a surgical wound, a trauma wound and a burn.

21. The method of claim 15, further comprising the step of refreshing the area with a fresh supply of the gas mixture.

22. The method of claim 15, wherein the step of refreshing utilizes an agitator.

23. The method of claim 15, wherein a jet of the gas mixture is delivered to the wound.

24. The method of claim 15, wherein the source of nitric oxide is a pressurized cylinder containing nitric oxide gas.

25. The method of claim 15, wherein the source of oxygen is a pressurized cylinder containing oxygen.

26. The method of claim 15, further comprising the step of monitoring the concentration of nitric oxide bathing the area.

27. The method of claim 15, further comprising the step of monitoring the concentration of nitrogen dioxide bathing the area.

28. The method of claim 15, further comprising the step of monitoring the concentration of oxygen bathing the area.

29. The method of claim 17, further comprising the step of evacuating the gas mixture from the area.

30. The method of claim 15, wherein the gas mixture is delivered to the wound at a pressure greater than 1 atmosphere.

31. A method to promote healing of a lesion, the method comprising the steps on identifying the lesion on the body of a patient; providing a flow-controlled source of gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; and delivering the gas mixture to the lesion on the body of the patient; and wherein the lesion is infected by at least one type of pathogen.

32. The method of claim 31, wherein the pathogen is at least one selected from the group consisting of a bacterium, a virus, a fungus, a parasite, an arthropod and a protozoan.

33. The method of claim 31, wherein the pathogen is an antibiotic-resistant bacteria.

34. The method of claim 33, wherein the antibiotic-resistant bacteria is at least one selected from the group consisting of Staphylococcus aureus, Pseudomonas aeruginosa, Streptococcus pneumoniae, Escherichia coli, Salmonella, Klebsiella, Enterococci, and combinations thereof.

35. The method of claim 31, further comprising the step of sealing the gas mixture.

36. The method of claim 31, further comprising the step of diluting the gas mixture.

37. The method of claim 31, wherein the nitric oxide gas flows from a pressurized cylinder containing nitric oxide gas.

38. The method of claim 31, wherein the oxygen gas flows from a pressurized cylinder containing oxygen gas.

39. The method of claim 31, further comprising the step of adjusting the pressure of the gas mixture delivered to the lesion.

40. The method of claim 31, further comprising evacuating the gas mixture at a flow rate substantially equal to a flow rate of the gas mixture delivered to the lesion.

41. The method of claim 31, wherein the lesion is a wound.

42. The method of claim 41, wherein the wound is at least one selected from the group consisting of a surgical wound, a trauma wound and a burn.

43. The method of claim 31, wherein the lesion is an abscess.

44. The method of claim 31, wherein the gas mixture is delivered to the lesion at a pressure greater than 1 atmosphere.

45. A method to promote healing of a lesion, the method comprising the steps of: identifying the lesion in the body of a patient; providing a flow-controlled source of gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; and delivering the gas mixture to the lesion in the body of the patient; and wherein the lesion is infected by at least one type of pathogen.

46. The method of claim 45, wherein the pathogen is at least one selected from the group consisting of a bacterium, a virus, a fungus, a parasite, an arthropod and a protozoan.

47. The method of claim 45, wherein the pathogen is an antibiotic-resistant bacteria.

48. The method of claim 47, wherein the antibiotic-resistant bacteria is at least one selected from the group consisting of Staphylococcus aureus, Pseudomonas aeruginosa, Streptococcus pneumoniae, Escherichia coli, Salmonella, Klebsiella, Enterococci, and combinations thereof.

49. The method of claim 45 further comprising the step of diluting the gas mixture.

50. The method of claim 45, wherein the source of nitric oxide gas is a pressurized cylinder containing nitric oxide gas.

51. The method of claim 45, wherein the source of oxygen gas is a pressurized cylinder containing oxygen gas.

52. The method of claim 45, further comprising the step of adjusting the pressure of the gas mixture delivered to the lesion.

53. The method of claim 45, further comprising evacuating the gas mixture at a flow rate substantially equal to a flow rate of the gas mixture delivered to the lesion.

54. The method of claim 45, wherein the lesion is a wound.

55. The method of claim 54, wherein the wound is at least one selected from the group consisting of a surgical wound, a trauma wound and a burn.

56. The method of claim 45, wherein the lesion is an abscess.

57. The method of claim 45, wherein the gas mixture is delivered to the lesion at a pressure greater than 1 atmosphere.

58. A method to promote healing of a lesion on or in the body of a patient, the method comprising the steps of: providing a source of gas containing nitric oxide and a source of a gas containing oxygen; mixing the gas containing nitric oxide with the gas containing oxygen, wherein the gas mixture contains at least about 10,000 ppm nitric oxide and at least about 20% oxygen; diluting the gas mixture; and delivering the diluted gas mixture to the lesion; wherein the lesion is infected by at least one type of pathogen.

59. The method of claim 58, wherein the pathogen is at least one selected from the group consisting of a bacterium, a virus, a fungus, a parasite, an arthropod and a protozoan.

60. The method of claim 58, wherein the pathogen is an antibiotic-resistant bacteria.

61. The method of claim 60, wherein the antibiotic-resistant bacteria is at least one selected from the group consisting of Staphylococcus aureus, Pseudomonas aeruginosa, Streptococcus pneumoniae, Escherichia coli, Salmonella, Klebsiella, Enterococci, and combinations thereof.

62. The method of claim 58, wherein the step of mixing the gas containing nitric oxide with the gas containing oxygen is performed using a gas blender.

63. The method of claim 58, wherein the gas mixture delivered to the lesion is flow controlled.

64. The method of claim 58, wherein the step of diluting the gas mixture further comprises the step of delivering a dilutant gas to a sealed area over the lesion.

65. The method of claim 58, further comprising the step of monitoring the concentration of nitric oxide being delivered.

66. The method of claim 65, further comprising the step of monitoring the concentration of oxygen being delivered.

67. The method of claim 58, wherein the lesion is a wound.

68. The method of claim 67, wherein the wound is at least one selected from the group consisting of a surgical wound, a trauma wound and a burn.

69. The method of claim 58, wherein the lesion is an abscess.

70. The method of claim 58, wherein the gas mixture is delivered to the lesion at a pressure greater than 1 atmosphere.

71. A method of treating an infected site by exposure to a gas mixture containing nitric oxide and oxygen, comprising the steps of providing a source of gas containing nitric oxide and a source of gas containing oxygen; mixing the gas containing nitric oxide with the gas containing oxygen, wherein the gas mixture contains at least about 10,000 ppm nitric oxide and at least about 20% oxygen; delivering the gas mixture to the infected site so as to bathe the infected site with the gas mixture; and wherein the infected site is infected by at least one type of pathogen.

72. The method of claim 71, wherein the pathogen is at least one selected from the group consisting of a bacterium, a virus, a fungus, a parasite, an arthropod and a protozoan.

73. The method of claim 71, wherein the pathogen is an antibiotic-resistant bacteria.

74. The method of claim 73, wherein the antibiotic-resistant bacteria is at least one selected from the group consisting of Staphylococcus aureus, Pseudomonas aeruginosa, Streptococcus pneumoniae, Escherichia coil, Salmonella, Klebsiella, Enterococci, and combinations thereof.

75. The method of claim 71, wherein the gas mixture is delivered to the infected site using a bathing unit surrounding the infected site.

76. The method of claim 71, further comprising the step of refreshing the infected site with a fresh supply of the gas mixture.

77. The method of claim 71, wherein a jet of the gas mixture is delivered to the infected site.

78. The method of claim 71, wherein the source of nitric oxide gas is a pressurized cylinder containing nitric oxide gas.

79. The method of claim 71, wherein the source of oxygen gas is a pressurized cylinder containing oxygen gas.

80. The method of claim 71, further comprising the step of monitoring the concentration of nitric oxide bathing the infected site.

81. The method of claim 71, further comprising the step of monitoring the concentration of oxygen bathing the infected site.

82. The method of claim 71, further comprising the step of monitoring the concentration of nitrogen dioxide bathing the infected site.

83. The method of claim 71, further comprising the step of evacuating the gas mixture from the area surrounding the infected site.

84. The method of claim 71, further comprising the step of stripping nitric oxide from the evacuated gas mixture.

85. The method of claim 71, further comprising the step of stripping nitrogen dioxide from the evacuated gas mixture.

86. The method of claim 71, wherein the infected site is a wound.

87. The method of claim 86, wherein the wound is at least one selected from the group consisting of a surgical wound, a trauma wound and a burn.

88. The method of claim 71, wherein the infected site is an abscess.

89. The method of claim 71, wherein the infected site is a lesion.

90. The method of claim 71, wherein the gas mixture is delivered to the infected site at a pressure greater than 1 atmosphere.

91. A method of treating an infected site by exposure to a gas mixture comprising the steps of: providing a source of gas containing nitric oxide and a source of gas containing oxygen; mixing the gas containing nitric oxide with the gas containing oxygen, wherein the gas mixture contains at least about 10,000 ppm nitric oxide and at least about 20% oxygen; delivering the gas mixture to the infected site so as to bathe the infected site with the gas mixture; evacuating the gas mixture from the area surrounding the infected site; and stripping nitric oxide from the evacuated gas mixture.

92. The method of claim 91, wherein the infected site is a wound.

93. The method of claim 92, wherein the wound is at least one selected from the group consisting of a surgical wound, a trauma wound and a burn.

94. The method of claim 91, wherein the infected site is an abscess.

95. The method of claim 91, wherein the infected site is a lesion.

96. The method of claim 91, wherein the gas mixture is delivered to the infected site at a pressure greater than 1 atmosphere.

97. A method of treating an infected site with a gas mixture comprising the steps of: providing a source of gas containing nitric oxide and a source of gas containing oxygen; mixing the gas containing nitric oxide with the gas containing oxygen, wherein the gas mixture contains at least about 10,000 ppm nitric oxide and at least about 20% oxygen; delivering the gas mixture to the infected site so as to bathe the infected site with the gas mixture; evacuating the gas mixture from the area surrounding the infected site; and stripping nitric dioxide from the evacuated gas mixture.

98. The method of claim 97, wherein the step of refreshing utilizes an agitator.

99. The method of claim 97, wherein the infected site is a wound.

100. The method of claim 99, wherein the wound is at least one selected from the group consisting of a surgical wound, a trauma wound and a burn.

101. The method of claim 97, wherein the infected site is an abscess.

102. The method of claim 97, wherein the infected site is a lesion.

103. The method of claim 97, wherein the gas mixture is delivered to the infected site at a pressure greater than 1 atmosphere.

104. A method of treating an infected site comprising the steps of: identifying the infected site on or in a human; providing a flow-controlled source of gas containing nitric oxide and a flow-controlled source of gas containing oxygen; mixing the gas containing nitric oxide with the gas containing oxygen, wherein the gas mixture contains at least about 10,000 ppm nitric oxide and at least about 20% oxygen; and delivering the gas mixture to at least a portion of the infected site.

105. The method of claim 104, further comprising the step of sealing the gas mixture.

106. The method of claim 104, further comprising the step of diluting the gas mixture with a dilutant gas.

107. The method of claim 104, wherein the flow-controlled source of nitric oxide gas flows from a pressurized cylinder containing nitric oxide gas.

108. The method of claim 104, further comprising the step of adjusting the pressure of the gas mixture delivered to the infected site.

109. The method of claim 104, further comprising evacuating the gas mixture at a flow rate substantially equal to a flow rate of the gas mixture delivered to the infected site.

110. The method of claim 104, wherein the step of refreshing utilizes an agitator.

111. The method of claim 104, wherein the infected site is a wound.

112. The method of claim 111, wherein the wound is at least one selected from the group consisting of a surgical wound, a trauma wound and a burn.

113. The method of claim 104, wherein the infected site is an abscess.

114. The method of claim 104, wherein the gas mixture is delivered to the infected site at a pressure greater than 1 atmosphere.

115. A method of treating an infected site by exposure to a gas mixture comprising the steps of: providing a source of gas containing nitric oxide and a source of gas containing oxygen; mixing the gas containing nitric oxide with the gas containing oxygen, wherein the gas mixture contains at least about 10,000 ppm nitric oxide and at least about 20% oxygen; diluting the gas mixture with a dilutant gas; delivering the diluted gas mixture to at least a portion of the infected site.

116. The method of claim 115, wherein the step of mixing the gas containing nitric oxide with the gas containing oxygen is performed using a gas blender.

117. The method of claim 115, wherein the gas mixture delivered to the infected site is flow controlled.

118. The method of claim 115, wherein the step of diluting the gas mixture further comprises the step of delivering the dilutant gas to a sealed area over the infected site.

119. The method of claim 115, further comprising the step of monitoring the concentration of nitric oxide being delivered.

120. The method of claim 115, further comprising the step of refreshing the gas mixture utilizing an agitator.

121. The method of claim 115, wherein the infected site is a wound.

122. The method of claim 121, wherein the wound is at least one selected from the group consisting of a surgical wound, a trauma wound and a burn.

123. The method of claim 115, wherein the infected site is an abscess.

124. A method of delivering a gas mixture to an infected site of a patient to reduce pathogen levels comprising the steps of: providing a flow-controlled source of a gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; providing a bathing unit around the infected site of the patient, the bathing unit forming a seal with the infected site of the patient; transporting the gas mixture to the bathing unit so as to bathe the infected site of the patient with the gas mixture; controlling the flow rate of the gas mixture into and out of the bathing unit; agitating the gas mixture within the bathing unit; directing the flow of gas mixture onto the infected site of the patient; actively or passively evacuating at least a portion of the gas mixture from the bathing unit; and removing at least a portion of the nitric oxide contained within the gas mixture that is evacuated from the bathing unit.

125. A method to promote healing of an area of the body of a patient with a wound, the method comprising the steps of: providing a flow-controlled source of a gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; and delivering the gas mixture to the area of the body so as to bathe the wound; wherein the gas mixture is delivered to the area using a bathing unit surrounding the area; wherein the gas mixture bathing the area is refreshed utilizing an agitator; wherein the wound is infected by at least one type of pathogen; wherein the source of gas containing nitric oxide is a pressurized cylinder containing nitric oxide gas; wherein the source of gas containing oxygen is a pressurized cylinder containing oxygen; wherein the concentration of nitric oxide bathing the area is monitored; wherein the concentration of nitrogen dioxide bathing the area is monitored; wherein the concentration of oxygen bathing the area is monitored; and wherein the gas mixture is evacuated from the area.

126. A method to promote healing of a lesion, the method comprising the steps of: identifying the lesion on the body of a patient; providing a flow-controlled source of a gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; and delivering the gas mixture to the lesion on the body of the patient; wherein the lesion is infected by at least one type of pathogen; wherein the delivery area of the gas mixture is sealed to prevent contact with air in the atmosphere; wherein the source of gas containing nitric oxide is a pressurized cylinder; wherein the source of gas containing oxygen is a pressurized cylinder; wherein the pressure of the gas mixture delivered to the lesion is adjusted; and wherein the gas mixture is evacuated at a flow rate substantially equal to the flow rate that the gas mixture is delivered to the lesion.

127. A method of treating an infected site by exposure to a gas mixture comprising the steps of: providing a flow-controlled source of a gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; and delivering the gas mixture to the infected site so as to bathe the infected site with the gas mixture; wherein the infected site is infected by at least one type of pathogen; wherein the gas mixture is delivered to the infected site using a bathing unit surrounding the infected site; wherein the gas mixture bathing the infected site is refreshed with a fresh supply of the gas mixture; wherein a jet of gas mixture is delivered to the infected site; wherein the source of gas containing nitric oxide is a pressurized cylinder; wherein the source of gas containing oxygen is a pressurized cylinder; wherein the concentration of nitric oxide bathing the infected site is monitored; wherein the concentration of oxygen bathing the infected site is monitored; wherein the concentration of nitrogen dioxide bathing the infected site is monitored; wherein the gas mixture bathing the infected site is evacuated; wherein nitric oxide is stripped from the evacuated gas mixture; and wherein nitrogen dioxide is stripped from the evacuated gas mixture.

128. A method of treating an infected site with a gas containing 10,000 ppm nitric oxide, in combination with 20% oxygen, comprising the steps of providing a flow-controlled source of a gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; delivering the gas mixture to the infected site so as to bathe the infected site with the gas mixture; evacuating the gas mixture from the area surrounding the infected site; stripping nitric dioxide from the evacuated gas mixture; and refreshing the gas mixture utilizing an agitator.

129. A method of treating an infected site comprising the steps of: identifying the infected site on or in a human; providing a flow-controlled source of a gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; delivering the gas mixture to at least a portion of the infected site; sealing the delivery area to prevent contact with air in the atmosphere; wherein the source of the gas containing nitric oxide is a pressurized cylinder; wherein the source of the gas containing oxygen is a pressurized cylinder; wherein the pressure of the gas mixture delivered to the infected site is adjusted; evacuating the gas mixture at a flow rate substantially equal to the flow rate of the gas mixture delivered to the infected site; refreshing the gas mixture bathing the infected site utilizing an agitator.

130. A method of treating an infected site by exposure to gas mixture comprising the steps of: providing a flow-controlled source of a gas containing nitric oxide and a flow-controlled source of a gas containing oxygen; mixing the gas containing nitric oxide with the gas containing oxygen, wherein the gas mixture contains at least about 10,000 ppm nitric oxide and at least about 20% oxygen; diluting the gas mixture; delivering the diluted gas mixture to at least a portion of the infected site; wherein the step of diluting the gas mixture is performed using a gas blender; wherein the gas mixture delivered to the infected site is flow controlled; wherein the step of diluting the gas mixture further comprises the step of delivering the gas mixture to a sealed area over the infected site; monitoring the concentration of nitric oxide being delivered; monitoring the concentration of oxygen being delivered; and refreshing the gas mixture bathing the infected site utilizing an agitator.
